(19) United States
(12) Patent Application Publication
(10) Pub. No.: US 2026/0108625 A1
(43) Pub. Date: Apr. 23, 2026

(54) BIOCOMPATIBLE NANOPARTICLES AND USES THEREOF

(71) Applicant: BAR-ILAN UNIVERSITY, Ramat Gan (IL)

(72) Inventors: Gerardo BYK, Tel-Aviv Yafo (IL); Eswaran LAKSHMANAN, Kiryat Ono (IL); Cyril Joseph COHEN, Petach-Tikva (IL); Maria RADMAN, Ashqelon (IL)

(21) Appl. No.: 19/117,610

(22) PCT Filed: Sep. 27, 2023
(86) PCT No.: PCT/IL2023/051042

§ 371 Date: Apr. 1, 2025

Related U.S. Application Data
(60) Provisional application No. 63/378,071, filed on Oct. 2, 2022.

(51) Int. Cl. A61K 48/00; A61K 9/06

(52) U.S. Cl. CPC A61K 48/0041; A61K 9/06

(57) ABSTRACT

A process for preparing polymeric monodispersed reduced nanohydrogels (NHGs) comprises: a) combining monomers, macromonomer, a cross-linker, and a surfactant to obtain a precursor mixture; b) heating the precursor mixture to a temperature of at least 40° C.; c) adding an initiator to the precursor mixture to obtain polymeric NHG; and d) performing an NHG reduction step. Polymeric monodispersed reduced NHGs and methods of use thereof are also disclosed.

### FIELD OF THE INVENTION

The present invention relates generally to the field of biocompatible nanoparticles and is directed to processes for preparing biocompatible nanoparticles and to methods of using the same for drug and nucleic acid (DNA or RNA) delivery and to express endogenous or exogenous proteins.

### BACKGROUND OF THE INVENTION

Polymeric nanoparticles with subcellular size can be loaded with “cargo” molecules (such as proteins, peptides, DNA, RNA, etc.) and be effectively endocytosed by cells resulting in high cellular uptake of the cargo, thereby serving as drug and nucleic acid delivery systems.
Effective bio-applicability of polymeric nanoparticles requires high biocompatibility, high stability in non-delivery areas, adjustable particle size, high cargo loading, reasonable circulation time, and short-term cargo release. Also, the polymeric nanoparticles must not aggregate since aggregation presents a risk of embolism due to capillary occlusion.

Gene therapy is designed to introduce genetic material into cells to compensate for abnormal genes, thereby generating a beneficial protein. If a mutated gene causes an essential protein to be faulty or missing, gene therapy may be able to introduce a normal copy of the gene to restore the function of the protein. Moreover, gene therapy has emerged in the last decade of the 20^{th} century as an essential technique to face genetically caused diseases, such as cancer, Alzheimer's disease, Parkinson's disease, and spinal muscular atrophy. There are various gene therapy clinical trials in progress for cancer treatment including the use of adenoviruses, adeno-associated virus, alphaviruses, herpes simplex virus and lentiviruses.

Polymeric monodispersed biocompatible nanohydrogels (NHGs) with a large range of discreet sizes starting from 20 nm up to 500 nm were previously described. However, known NHGs suffer some limitations. First, they are stable only in aqueous organic medium, and are irreversibly transformed into a film or gel upon drying, losing their nanoparticle properties. Additionally, their negative zeta potential prevents their complexation to nucleic acids (DNA, RNA).

For the above and other purposes there is a need for new stable biocompatible NHGs and processes for the preparation thereof.

### SUMMARY OF THE INVENTION

A process for preparing polymeric monodispersed reduced nanohydrogels (NHGs) comprising:
a) combining monomers, macromonomer, a cross-linker, and a surfactant to obtain a precursor mixture;
b) heating the precursor mixture to a temperature of at least 40° C.;
c) adding an initiator to the precursor mixture to obtain polymeric NHG; and
d) performing an NHG reduction step.

In one embodiment, the cross-linker is bis-acrylamide (BIS). In another embodiment, the surfactant is Polyvinylpyrrolidone (PVP).

In a particular embodiment of the process of the invention the reduction step comprises:
a) preparing a magnetic embedded NHG magnetite or maghemite matrix by combining an NHG with FeCl₃·6 H₂O and FeCl₂·4 H₂O to obtain a reaction mixture, and adding ammonium hydroxide thereto;
b) reducing said magnetic embedded NHG matrix by combining it with an organic solvent-soluble reducing agent to obtain magnetic embedded NHG; and
c) releasing the NHG by dissolving the magnetic matrix in an acid.

In one embodiment, the organic solvent-soluble reducing agent is selected from boric acid, trimethyl borate, and borane-THF complex. The monomers and/or the macromonomer can be composed of different chains of jeffamines linked to polymerization groups. In some embodiments, the polymerization groups are selected from acrylamides, metacrylamides or other substituted acrylamides, or N-isopropyl acrylamide (NIPA), N-diisopropyl acrylamide, N-diisopropyl metacrylamide, acrylonitrile, and any combination thereof.

The invention also encompasses a polymeric monodispersed reduced NHG, whenever prepared by the process of the invention. Said polymeric monodispersed reduced NHG may have, for instance, a monodispersed size of about 20 nm to about 500 nm, preferably from about 50 nm to about 400 nm.

An embodiment of the process for preparing a polyplex comprising the NHG and one or more nucleic acids, comprises adding one or more nucleic acids to the reduced NHG obtained by the process of the invention. Similarly, the invention provides for the preparation of a polyplex comprising a polymeric reduced monodispersed NHG and one or more nucleic acids.

In another aspect, the invention is directed to the use of a polyplexes according to the invention for transferring genes into mammalian cells to express endogenous or exogenous proteins, as well as to a method for the delivery of endogenous or exogenous proteins to the brain or heart comprising using a polyplex of the invention and immune cells as nucleic acid shuttles. Furthermore, another aspect of the invention provides a method using polyplexes of the invention to transfer genes into mammalian cells to express an endogenous or exogenous protein, for a use selected from vaccination, treatment of genetic diseases such as hemophilia, cystic fibrosis, spinal muscular atrophy (SMA), Duchesne Myopathy and other genetic diseases, or cancer treatment.

Other aspects, purposes and advantages of the invention will become apparent as the description proceeds.

### BRIEF DESCRIPTION OF THE DRAWINGS

FIG. 1 shows TGA curves of magnetic matrix embedded 50 nm NHGs;

FIG. 2 shows FTIR spectra of the NHGs. Lines 1 and 2 show 400 nm NHGs before and after reduction, respectively; Lines 3 and 4 show 200 nm NHGs before and after reduction, respectively; Lines 5 and 6 show 50 nm NHGs before and after reduction, respectively;

FIGS. 3A, 3B, and 3C show topography image of the 400 nm reduced NHGs at 1, 0.1, and 0.01 mg/ml respectively on the wafer;

FIGS. 4A, 4B, and 4C show topography images of the 200 nm reduced NHGs at 1, 0.1, and 0.01 mg/ml respectively on the wafer;

FIGS. 5A, 5B, and 5C show gel electrophoresis assay with various amounts of reduced 400 nm, 200 nm and 50 nm NHGs respectively with GFP plasmid;

FIG. 6A shows proliferation of HEK293T cells after 2 days incubation with different sizes of polyplexes (400 nm, 200 nm and 50 nm) and lipoplexes. A—Naked NHGs (20 μg), B—NHGs/DNA (20 μg/0.5 μg), C—Naked DNA (0.5 μg), D—Lipid/DNA (6 nmol/0.5 μg), E—Triton. Each point represents the mean value ±SD (n=2);

FIG. 6B shows proliferation of HEK293T cells after 6 days incubation with different sizes of polyplexes (400, 200 nm and 50 nm) and lipoplexes. A—Naked NHGs (20 μg), B—NHGs/DNA (10 μg/0.5 μg), C—Naked DNA (0.5 μg), D—Lipid/DNA (6 nmol/0.5 μg), E—Triton. Each point represents the mean value ±SD (n=2);

FIG. 7 shows fluorescent microscopic images (10×) of HEK293T cells after 24 h incubation with polyplexes. A1—control cells, A2—12.5 μg NHGs, A3—0.5 μg DNA, A4—Lipid/DNA (6 nmol/0.5 μg), A5—NHG/DNA (12.5 μg/0.5 μg), A6—L-NHG/DNA (12.5 μg/0.5 μg). Scale bar: 100 μm;

FIG. 8 shows fluorescent microscopic images (10×) of HEK293T cells after 48 h incubation with polyplexes. B1—control cells, B2—12.5 μg NHGs, B3—0.5 μg DNA, B4—Lipid/DNA (6 nmol/0.5 μg), B5—NHG/DNA (12.5 μg/0.5 μg), B6—L-NHG/DNA (12.5 μg/0.5 μg). Scale bar: 100 μm;

FIG. 9 shows fluorescent microscopic images (10×) of HEK293T cells after 6 days incubation with polyplexes. C1—control cells, C2—12.5 μg NHGs, C3—0.5 μg DNA, C4—Lipid/DNA (6 nmol/0.5 μg), C5—NHG/DNA (12.5 μg/0.5 μg), C6—L-NHG/DNA (12.5 μg/0.5 μg). Scale bar: 100 μm;

FIG. 10 shows fluorescent microscopic images (10×) of Hela cells after 6 days incubation with polyplexes ( ). A1—control cells, A2—Lipid/DNA (6 nmol/0.5 μg), A3—NHG/DNA (5 μg/0.5 μg), A4—NHG/DNA (10 μg/0.5 μg) A5—NHG/DNA (12.5 μg/0.5 μg). Scale bar: 100 μm;

FIGS. 11A and 11B show comparative fluorescence microscopic images (10×) of HEK293T cells after 6 days incubation with 400 nm polyplexes. ( ). A1—control cells, A2—200 nm NHG/salmon DNA (12.5 ug/0.5 ug), A3—lipid/GFP (6 nmol/0.5 ug), A4—200 nm NHG/GFP (12.5 ug/0.5 ug), A5—400 nm NHG/salmon DNA (12.5 ug/0.5 ug), A6—400 nm NHG/GFP (1 ug/0.5 ug), A7—400 nm NHG/GFP (2.5 ug/0.5 ug), A8—400 nm NHG/GFP (5 ug/0.5 ug), A9—400 nm NHG/GFP (10 ug/0.5 ug), A10-400 nm NHG/GFP (12.5 ug/0.5 ug). Scale bar: 100 μm;

FIG. 12 shows fluorescence microscopic images (10×) of HEK293T cells after 6 days incubation with 50 nm polyplexes. A1—control cells, A2—lipid/GFP (6 nmol/0.5 μg), A3—200 nm NHG/GFP (12.5 μg/0.5 μg), A4—50 nm NHG/GFP (5 μg/0.5 μg), A5—50 nm NHG/GFP (10 μg/0.5 μg), A6—50 nm NHG/GFP (12.5 μg/0.5 μg). Scale bar: 100 μm;

FIG. 13 shows fluorescent microscopic images (10×) of HEK293T cells incubated with 200 nm polyplexes. A1—NHG/GPF (12.5 μg/0.5 μg) after 48 h incubation, A2—only cells, A3—NHG/GPF (12.5 μg/0.5 μg) two days after passaging. Scale bar: 100 μm;

FIG. 14 shows fluorescent microscopic images (10×) of HEK293T cells incubated with 200 nm polyplexes. B1—only cells, B2—NHG/GPF (12.5 μg/0.5 μg) 4 days after passaging. Scale bar: 100 μm;

FIG. 15 shows fluorescent microscopic images (10×) of HEK293T cells incubated with 200 nm polyplexes. C1—only cells, C2—NHG/GPF (12.5 μg/0.5 μg) 7 days after passaging. Scale bar: 100 μm;

FIG. 16 shows fluorescent microscopic images (10×) of HEK293T cells incubated with 200 nm polyplexes. D1—only cells, D2—NHG/GPF (12.5 μg/0.5 μg) 9 days after passaging. Scale bar: 100 μm;

FIG. 17 shows fluorescent microscopic images (10×) of HEK293T cells incubated with 200 nm polyplexes. E1—only cells, E2—NHG/GPF (12.5 μg/0.5 μg) 11 days after passaging. Scale bar: 100 μm;

FIG. 18 shows fluorescent microscopic images (10×) of HEK293T cells incubated with 200 nm polyplexes. F1—only cells, F2—NHG/GPF (12.5 μg/0.5 μg) 14 days after passaging. Scale bar: 100 μm;

FIGS. 19A, 19B, and 19C show imaging using Incucyte system (10×) of HEK293T, Hela and NIH-3T3 cells, respectively, after 1 week incubation with polyplexes. A1—control cells, A2—lipid/GFP (6 nmol/0.5 μg), A3—200 nm NHG/GPF (12.5 μg/0.5 μg), A4—400 nm NHG/GPF (2.5 μg/0.5 μg), A5—400 nm NHG/GPF (5 μg/0.5 μg), A6—400 nm NHG/GPF (10 μg/0.5 μg), A7—400 nm NHG/GPF (12.5 μg/0.5 μg), A8—50 nm NHG/GPF (10 μg/0.5 μg), A9—50 nm NHG/GPF (12.5 μg/0.5 μg), A10—50 nm NHG/GPF (15 μg/0.5 μg), A11—50 nm NHG/GPF (20 μg/0.5 μg).Scale bar: 400 μm, area: 1.75×1.29 mm, 2.27 mm²;

FIG. 20 shows quantification of fluorescence obtained from Incucyte microscope of HEK293T, Hela and NIH-3T3 cells after 6 days incubation with polyplexes. A1—control cells, A2—lipid/GFP (6 nmol/0.5 μg), A3—200 nm NHG/GPF (12.5 μg/0.5 μg), A4—400 nm NHG/GPF (2.5 μg/0.5 μg), A5—400 nm NHG/GPF (5 μg/0.5 μg), A6—400 nm NHG/GPF (10 μg/0.5 μg), A7—400 nm NHG/GPF (12.5 μg/0.5 μg), A8—50 nm NHG/GPF (10 μg/0.5 μg), A9—50 nm NHG/GPF (12.5 μg/0.5 μg), A10—50 nm NHG/GPF (15 μg/0.5 μg), A11—50 nm NHG/GPF (20 μg/0.5 μg). Total area for each picture: 2.27 mm²;

FIGS. 21A and 21B show detection of m-Cherry 5, 12, 19, and 29 days after intramuscular administration of high concentration (40 μg) DNA (FIG. 21A) and low concentration (20 μg) DNA (FIG. 21B);

FIGS. 22A and 22B show detection of m-Cherry 5, 12, 19, and 29 days after subcutaneous administration of high concentration (40 μg) DNA (FIG. 22A) and low concentration (20 μg) DNA (FIG. 22B);

FIG. 23 shows injection sites after 29 days of intramuscular injection (left column) and subcutaneous injection (right column). H1-H11: administration of high concentration (40 μg) mCherry, L1-L11: administration of low concentration (20 μg) mCherry;

FIGS. 24A and 24B show brain and heart, respectively, 29 days after intramuscular injection (left column) and subcutaneous injection (right column) of mCherry;

FIG. 25 shows the average signal in brain, heart, liver, lung, and kidney tissues 29 days after intramuscular (IM) and subcutaneous (SC) injections. Each bar represents a single animal (animal number *) or control (C*);

FIG. 26A-26D show histological analysis of injected muscle. In FIGS. 26A and 26B eosin color shows inflammatory cells in the injected tissue. FIGS. 26C and 26D show inflammatory cells expressing mCherry (FIG. 26C) and DAPI (FIG. 26D);

FIG. 26A shows focal extensive infiltrated lesion composed of mineralized material (arrows 2 and 3) surrounded by inflammatory cells (arrow 1) in a striated muscle (sample #01);

FIG. 26B shows focal extensive infiltrated lesion composed of mineralized material surrounded by macrophages (arrow 1), multinucleated giant cells (arrow 4), lymphocytes (arrow 2), and neutrophils (arrow 3) in a striated muscle (sample #01);

FIG. 26C shows focal extensive infiltrated lesion composed of mineralized material (arrow 1) that is partly fluorescent positive surrounded by inflammatory cells (arrow 2) that are positive ×10 IF in a striated muscle (sample #01);

FIG. 26D shows DAPI staining of all nuclear nuclei in the striated muscle of sample #01 ×10 IF;

FIGS. 27A-27F show histological analysis of injected subcutaneous tissue. In FIGS. 27A and 27B eosin color shows inflammatory cells in the injected tissue. FIGS. 27C and 27E show inflammatory cells expressing m-cherry. FIGS. 27D and 27F show inflammatory cells expressing DAPI;

FIG. 27A shows focal extensive granuloma composed of mineralized material (arrows 1 and 2) surrounded by inflammatory cells (arrow 3) X4 H&E in a skin tissue of sample #02;

FIG. 27B shows focal extensive granuloma composed of mineralized material surrounded by macrophages (arrow 1), multinucleated giant cells (arrow 3), lymphocytes (arrow 4), and neutrophils (arrow 2) in a skin sample ×10 H&E (sample #02);

FIG. 27C shows focal extensive granuloma composed of mineralized material (arrows 1 and 2) that is partly fluorescence positive, surrounded by positive inflammatory cells (arrow 3) X10 IF in a skin sample (sample #02);

FIG. 27D shows DAPI staining of all nuclear nuclei in the skin sample ×10 IF (sample #02);

FIG. 27E shows focal extensive granuloma composed of mineralized material (arrow 2) that is partly fluorescence positive, surrounded by positive inflammatory cells (arrow 1) X20 IF in a skin sample (sample #02);

FIG. 27F shows DAPI staining of all nuclear nuclei in the skin sample ×20 IF (sample #02);

FIGS. 28A-28E show histological analysis of brain after IM administration. In FIGS. 28A and 28B eosin color shows inflammatory cells in the meninges area. FIGS. 28C and 28D show inflammatory cells expressing m-cherry (FIG. 28C) and DAPI (FIG. 28D). FIG. 28E shows merging of FIGS. 28C and 28D. Red fluorescent cells not seen with DAPI are erythrocytes.

FIG. 28A shows focal area of suspected macrophages and fewer lymphocytes (arrows 1 and 2) surrounded by meningeal endothelial cells (arrow 3) in mid-brain meninges ×10 H&E (sample #1M-01);

FIG. 28B shows focal area of suspected macrophages (arrow 1) and fewer lymphocytes (arrow 2) surrounded by meningeal endothelial cells (arrow 3) in mid-brain meninges ×20 H&E (sample #IM-01);

FIG. 28C shows focal area of suspected inflammatory cells (arrow 1) in mid brain meninges ×10 IF (sample #IM-01). Auto-fluorescence is due to many erythrocytes;

FIG. 28D shows DAPI staining in focal area of suspected inflammatory cells (arrow 1) in mid brain meninges ×10 (sample #IM-01);

FIGS. 29A-29D show histological analysis of heart after IM administration;

FIG. 29A shows inflammatory cells (H&E color);

FIG. 29B shows cells expressing m-cherry;

FIG. 29C shows DAPI staining;

FIG. 29D shows merging of FIGS. 29B and 29C. Red fluorescent cells not seen with DAPI are erythrocytes;

FIG. 30 shows the expression of mRNA along 11 days incubation of HEK293T cells with the complexes containing m-Cherry mRNA, as measured with IncuCyte microscope. After day 7 cells were passed and measurement was continued until day 11;

FIGS. 31A-31C show protein expression in HEK293T cells at the end of the experiment (day 11). FIG. 31A shows cells only (control), FIG. 31B shows cells incubated with 12.5 μg mRNA, and FIG. 31C shows cells incubated with 5 μg mRNA.

### DETAILED DESCRIPTION OF THE INVENTION

The present invention relates to biocompatible particles or nanoparticles as well as a process for their preparation. The nanoparticles of the present invention are highly biocompatible and may comprise complex nucleic acids (DNA, RNA, etc.) to result in nanocomplexes having the same size as the original nanoparticles. The nanocomplexes of the present invention can transfer genes into mammalian cells, resulting in the expression of endogenous or exogenous proteins or silencing of endogenous proteins. The complexes can be incubated with cells for a non-limited duration without toxicity. As used herein the term “nucleic acid” refers to DNA and/or RNA.

In some embodiments the present invention provides a process for preparing polymeric monodispersed reduced nanohydrogels (NHGs) comprising:
a) combining monomers, macromonomer, cross-linker, and surfactant, to obtain a precursor mixture;
b) heating the precursor mixture preferably to a temperature of at least 40° C.;
c) adding to the precursor mixture an initiator to obtain polymeric NHG; and
d) performing an NHG reduction step.

Typically, the monomers and/or macromonomers are composed of different chains of jeffamines linked to polymerization groups such as acrylamides, metacrylamides or other substituted acrylamides. Alternatively, the monomers and/or macromonomers are selected from N-diisopropyl acrylamide, N-diisopropyl metacrylamide, acrylonitrile, and any combination thereof.

The ratio between monomers and macromonomers predetermines the specific size of the nanoparticles.

The reduction step may comprise combining the polymeric NHG with an organic solvent-soluble reducing agent such as boric acid and borane-THF complex; centrifuging; washing and drying. Alternatively, the reduction step may comprise the following steps:
a) preparing a magnetic embedded NHG magnetite or maghemite matrix by combining NHG with FeCl₃·6 H₂O and FeCl₂·4 H₂O to obtain a reaction mixture; and adding ammonium hydroxide to obtain the magnetic embedded NHG matrix;
b) reducing the magnetic embedded NHG matrix by combining the magnetic NHG with an organic solvent-soluble reducing agent such as boric acid, trimethyl borate and borane-THF complex, to obtain magnetic embedded NHG; and
c) releasing the NHG by dissolving the magnetic matrix with an acid such as HCl.

In another embodiment, the present invention provides polymeric monodispersed reduced NHG, which is prepared by the processes described above.

The size of the NHG is predetermined by the macromonomer/monomer ratio, such that lower ratio results in larger NHGs. Typically, the nanoparticles of the present invention have mono-dispersed size of about 20 nm to about 500 nm, or from about 50 nm to about 400 nm, for example of 200 nm.

The NHGs of the present invention are characterized by negative zeta potential before reduction and positive zeta potential after reduction.

As discussed above, the reduced NHGs of the present invention may complex nucleic acids to form polyplexes. The present invention further provides a process for preparing polyplexes comprising the NHGs of the present invention and nucleic acids.

The process for preparing the polyplexes comprises preparing reduced NHG, for example by the process of the present invention, and adding nucleic acids, for example pDNA, mRNA or CRISPR/Cas9.

In yet another embodiment, the present invention provides polyplexes comprising polymeric reduced monodispersed NHG and nucleic acids, which are prepared by the processes described above.

Like the NHGs, the complexes of the present invention also have monodispersed size of about 20 nm to about 500 nm, or from about 50 nm to about 400 nm, for example of 200 nm. Both the NHGs and the complexes of the present invention are not toxic.

In one embodiment, the present invention provides use of the NHGs/nucleic acid complexes (DNA or RNA) of the present invention to transfer genes and/or mRNA into mammalian cells to express endogenous or exogenous proteins or silencing endogenous proteins.

Preferably, NHGs of 200 nm are used, with a ratio of 1-20 μg NHGs/0.5 μg nucleic acid, for example NHGs/DNA ratio of 12.5 μg NHGs/0.5 DNA. Alternatively, if NHGs of 400 nm are used, the ratio of NHGs/nucleic acid is 1-2.5 μg NHGs/0.5 μg nucleic acid, for example NHGs/DNA ratio of 2.5 μg NHGs/0.5 DNA.

In another embodiment, the present invention provides a method for transferring genes and/or mRNA into mammalian cells to express endogenous or exogenous proteins using the NHGs/nucleic acid complexes of the present invention.

In another embodiment, the present invention provides a method for local delivery of nucleic acids for expressing endogenous or exogenous proteins using the polyplexes of the present invention.

The transfer and/or delivery of the immunogenic proteins, DNA and/or RNA using the NHGs/nucleic acid complexes of the present invention, may be further used for vaccination, for treatment of genetic diseases such as hemophilia, cystic fibrosis, Duchesne Myopathy and other genetic diseases, or for cancer treatment.

Typically, when polyplexes are administered to animals, the transfected cells are mostly those of the immune system. The present invention further comprises a method for delivery of endogenous or exogenous proteins to the brain or heart using immune cells as nucleic acid shuttles.

In yet another embodiment, the present invention provides methods for a continuous and long release of a DNA plasmid, leading to sustained expression of the endogenous or exogenous protein at the site of administration or at the organs to which the nucleic acid polyplexes are transported (e.g., the brain and the heart).

Having described the invention with reference to certain preferred embodiments, other embodiments will become apparent to one skilled in the art from the specification. The invention is further defined by reference to the following examples describing in detail the preparation of compositions and methods of use of the invention. It will be apparent to those skilled in the art that many modifications, both to materials and methods, may be practiced without departing from the scope of the invention.

### EXAMPLES

### 1. Materials and Method

### 1.1. Synthesis of NHGs Containing Highly Reducible Groups

5 mg of BIS (0.03 mmol), 5 mg of PVP₃₆₀₀₀₀, 170 mg of N-isopropyl acrylamide (NIPAAM) (1.66 mmol), 60 mg acrylonitrile (11 mmol) and an appropriate amount of (Acr)1.1Jeffamine1900 for obtaining the desired NHGs size, were introduced into a 20 ml scintillation vial and fully dissolved in 8 ml DDW. The vial content was flushed with nitrogen and shaken at 73° C. for 1 h in a DAIHAN scientific shaking water bath. Then, a solution of KPS (5 mg, 0.018 mmol) in 2 ml DDW was added to the reaction mixture and the polymerization process was allowed to continue at 73° C. for 23 hours. The resulting NHG dispersion was allowed to cool to room temperature and dialyzed against 25 liters DDW for 1 week using Cellulose Ester Spectra/Pore 1000 kDa cut-off molecular weight dialysis membrane. The water was changed twice per day. Aliquots of purified NHGs dispersion were lyophilized to determine polymerization yield.

### 1.2. Reduction of NHGs (Method A)

The centrifuged polymeric NHGs (160 mg) and 319 mg boric acid (5.16 mmol) were taken in a glass tube. 607 μl of trimethyl borate (5.16 mmol) was added, followed by addition of 17.2 ml 1 M borane-THF complex (17.2 mmol). After cessation of hydrogen evolution, the tubes were capped tightly and kept in an oil bath at 65° C. for 72 h. The NHGs were then centrifuged, washed with MeOH (10 mL×2), suspended in methanol (8 mL) and piperidine (100%, 2 mL) and heated at 65° C. for 20 h to destroy the borane complexes. Following the decantation of the piperidine-borane solution, the NHGs were centrifuged and washed with distilled water (10 mL×3). Finally, the reduced NHGs were allowed to dialyze against 25 L DDW for 2 days (the DDW was replaced twice a day) using Cellulose Ester Spectra/Pore 1000 kDa cut-off molecular weight dialysis membrane.

### 1.3. Reduction of NHGs (Method B)

### 1.3.1. Preparation of the Magnetic Embedded NHGs Matrix

25 ml of 50 nm NHGs aqueous dispersion (about 200 mg NHGs) were added to 1 L three-necked bottom round flask and cooled in a water bath under N₂ gas bubbling for 45 min under vigorous stirring. 200 mg FeCl₃·6 H₂O (0.8 mmol) and 140 mg FeCl₂·4 H₂O (0.6 mmol) were dissolved in 3 ml DDW. Then the iron ions solution was combined and added to the flask containing NHGs. A light-yellow color mixture was formed. The ice bath was removed, and the flask was continuously evacuated under vacuum while the mixture kept stirring until no further foaming was observed in the mixture. The evacuation was stopped and the flask was immersed in a pre-heated water bath at 85° C. 5 ml of ammonium hydroxide (30%) were added. The reaction mixture turned gradually to brown. The mixture was stirred at 85° C. for 1.5 h after which it was cooled to room temperature. The resulting magnetic matrix was washed extensively with water (20 ml×5), DMF (20 ml×5) methanol (20 ml×5) and diethyl ether (20 ml×5) using an external magnetic field for separation to obtain 350 mg of magnetic matrix embedded 50 nm NHGs. A magnetic field was applied using an N50 magnet for several minutes, resulting in complete retainment of the magnetic matrix embedded NHGs. Finally, the magnetic matrix embedded NHGs were analyzed by TGA.

### 1.3.2. Reduction of Magnetic Embedded NHGs

40 mg magnetic NHGs (50 nm) and 79 mg boric acid (1.27 mmol) were mixed in a glass tube. 150 μl trimethyl borate (1.27 mmol) was added, followed by 4.25 ml 1 M borane-THF complex (4.25 mmol). After cessation of hydrogen evolution, the tubes were capped tightly and kept in an oil bath at 65° C. for 96 h. Next, The NHGs were centrifuged, washed with MeOH (20 mL×5), DMF (20 mL×5) and water (20 mL×5). The resulting NHGs were kept in 10 ml H₂O. Finally, the reduced NHGs were allowed to dialyze against 25 L DDW for 2 days (the DDW was replaced twice a day) using Cellulose Ester Spectra/Pore 1000 kDa cut-off molecular weight dialysis membrane.

### 1.3.3. Releasing the NHGs by Dissolving the Magnetic Matrix

8 ml HCl (5 M) was added to the matrix (40 mg) and stirred for 6 h to dissolve the magnetic matrix. The full dissolution of the matrix and regeneration of NHGs were monitored by DLS. Then, 200 mg EDTA tetrasodium salt (0.52 mmol) was added to the acidic dispersion of NHGs containing iron salt ions. The resulting mixtures were neutralized slowly to pH=6 by adding 30% NH₄OH. Then, the mixture was sonicated for several minutes and dialyzed against 25 L DDW for 2 days (the DDW was replaced twice a day) to obtain reduced NHGs.

### 1.4. Dynamic Light Scattering (DLS) and Zeta Potential

Hydrodynamic sizes, size distributions and zeta potential measurements were determined using a Zetasizer 3000 HSA (Malvern instruments Ltd., UK) operating with a 4 mW HeNe laser (632.8 nm), a detector positioned at a scattering angle of and a temperature-controlled jacket for the cuvette. For hydrodynamic sizes and size distributions measurements, each sample cuvette was left for 5 min at 25° C. and 10 min at 45° C. to allow temperature equilibration before measurement. Three to five measurements consisting of up to 10 consecutive sub-runs were performed for each sample and temperature. For the zeta potential measurements, 2 ml of 1 mg/ml NHGs dispersion (pH=7.4) were injected to the Zetasizer 3000 HSA pump. Three measurements consisting of 10 sub-runs were performed for each sample at 25° C. and 45° C.

### 1.5. Preparation of Polyplexes for Size Measurement

Polyplexes were prepared by mixing reduced NHGs and pDNA. To prepare polyplexes at NHGs/DNA ratio (W/W) (40:1, 20:1, 10:1, 5:1, 1:1 and 1:10), 1 ml NHG solution (500 g) was mixed with 2.5 mL DDW and then added into 20 μL of 12.5 μg pDNA solution to obtain a 40:1 ratio. In a similar way, 12.5 μg pDNA was used for all the other ratios with the appropriate amount of NHG's. Then the samples were mixed by gently pipetting and incubated at room temperature for 20 min to ensure polyplexes formation. Polyplexes sizes were measured by dynamic light scattering (DLS) using a Zetasizer 3000 HSA (Malvern instruments Ltd., UK)

### 1.6. Preparation of Polyplexes for Zeta Potential

To prepare polyplexes at NHG/DNA (W/W) ratio of 40:1, 1 mL of NHG (1 mg) solution was mixed with 1 mL PBS (pH-7.4) and then added into 1 ml of diluted pDNA (25 μg) solution to obtain a 40:1 ratio. In the same way, 25 μg pDNA was used for all other ratios with appropriate amounts of NHG. The samples were mixed slightly and incubated at room temperature for 20 min to ensure polyplexes formation. Polyplexes zeta potentials were measured by dynamic light scattering (DLS) using a Zetasizer 3000 HSA (Malvern instruments Ltd., UK).

### 1.7. Fourier-Transform Infrared Spectroscopy (FTIR)

The FTIR spectra were recorded on Thermo Nicolet iS10 with smart omni transmission spectrometer. The spectra were acquired (32 scans per sample or background) in the range of 4000-500 cm⁻¹. The spectra were corrected using the background spectrum of air. The analysis was carried out at room temperature. For a measurement, a lyophilized sample was dissolved with nujol mull and placed onto the surface of the NaCl crystal. Before acquiring a spectrum, the NaCl crystal was carefully cleaned with Kim wipes and dried.

### 1.8. Atomic Force Microscopy (AFM)

The AFM samples were prepared by simple spin coating method, different concentrations (1, 0.1 and 0.01 mg/mL NHGs, 50 μL) of NHGs were coated on a silicon wafer substrate. The spin coating timer was adjusted for 30 sec at 2000 RPM. After spin-coating samples were thoroughly dried out with nitrogen and transferred for (Bruker, AXS) AFM imaging.

### 1.9. Gel Electrophoresis

To prepare each complex, 0.5 μg of GFP was incubated for 20 minutes with varying amounts of NHG. The complexes were mixed with 5 μl DNA loading dye and the complexes were loaded on a 0.8% agarose gel (400 mg) containing 5 μl Gel-Red. The electrophoresis study was performed at 100 V for 40 min in a TBE buffer medium and then the bands were monitored using a UV trans-illuminator.

### 1.10. Cell Culture

NIH-3T3, HeLa and HEK-293T cells were cultured in DMEM medium and supplemented with 10% fetal bovine serum (FBS, Sigma-Aldrich NY, USA), 100 μg/mL streptomycin and 100 U/mL penicillin at 37° C. in 5% C02.

### 1.11. Cell Viability Assays

The viability of the cells was analysed by XTT assay. The cells were seeded with their media in 96-well flat-bottom plates at a density of 8×10³ cells per well and cultured in a humidified incubator (Water-Jacketed, US Autoflow Automatic CO₂ Incubator manufactured by NuAire, Inc.,) at 37° C. for 24 hours. Then, the media was removed by aspiration and 90.1 μl of fresh media were added. The cells were treated with 20 μg of naked NHG in 100 μL DMEM, polyplexes at ratio NHG/DNA 40:1 (20 μg NHG/0.5 μg DNA), 6 nmol/0.5 μg lipoplexes in 100 μL DMEM, 0.5 μg DNA in 100 μL DMEM, and 2% triton in 100 μL DMEM. Briefly, a dose of polyplexes/lipoplexes in media was added to each well (in quadruplicate) and incubated for 48 h. One row was used as control cells without the addition of NHG and one row containing only the appropriate medium was used for blank absorbance readings. Subsequently, 50 μL XTT reagent (with initiator) were added to the cells and incubated for 4 hours. The cells absorbance was read at 570 nm in a TECAN microplate reader. Cell viability % was calculated as follows: (Isample/Icontrol)×100, where Isample is the absorbance of NHG-treated wells and Icontrol is the absorbance of control wells without NHG treatment. These experiments were repeated twice.

### 1.12. Plasmid Preparation

The plasmids used in this work were GFP and mCherry. The plasmid DNA was amplified in E. coli and purified according to the supplier's protocol (Promega, USA). The quantity and quality of the purified plasmid DNA was assessed by optical density at 260 and 280 nm and by electrophoresis in 0.8% agarose gel.

### 1.13. In-Vitro Transfection

The cells were cultured (without antibiotics) in a 96-well plate at density of 5000 cells/well and incubated for 24 hours to obtain 70-80% of confluence prior to the addition of polyplexes/lipoplexes. Before the transfection, the medium was replaced with 100 μl of fresh medium (without serum). The vector/DNA complexes were prepared as follows: 0.5 μg DNA/well mixed in 40 μl DMEM and appropriate amount of lipid/NHGs mixed in 40.1 μl DMEM contain no serum. After 5 minutes incubation at room temperature, the diluted DNA was combined with diluted lipid/NHGs (total volume 80 μl) and incubated for 20 minutes at RT). The complexes were added to each well containing 100 μl of medium without serum and mixed gently by rocking the plate back and forth. After 4 h incubation at 37° C. in CO₂ incubator, 10% FBS was added. Finally, the complexes were incubated for 6 days prior to testing for transgene expression.

### 1.14. In-Vitro Transfection (Passing Method)

Polyplexes with the cells were passed and divided into 6 wells after one week of transfection. The wells were analyzed one by one at 48 h time-intervals starting from day 8 after incubation with polyplexes. Untreated cells were passed similarly to controls.

### 1.15. In-Vivo Analysis

7-8 weeks old BALB/c mice were purchased from ENVIGO ISRAEL. All animal experiments were reviewed and approved by Bar-Ilan University and all protocols met the requirements of the local ethical committee of the Bar-Ilan University, Israel. All mice had ad libitum access to food and water and were monitored throughout the total duration of the experiment. Two concentrations of mCherry DNA were used to prepare the polyplexes complexes (40 and 20 μg). In order to complex 40 and 20 μg of DNA, 1 mg and 0.5 mg of reduced NHGs were used, respectively. The polyplexes were injected to mice intramuscularly (IM, n=6) and subcutaneously (SC, n=5), wherein high concentration (1 mg NHGs with 40-ug mCherry) was injected to the left side of the animal's body and low concentration (0.5 mg NHGs with 20 μg mCherry) was injected to its right side. One mouse was used as control. The experiment was conducted over a period of 29 days. Fluorescence was studied by the Maestro II in vivo imaging system, 2D planar fluorescence imaging of small animals (Cambridge Research and Instrumentation (CRi), Inc., Woburn, MA). A yellow excitation/emission filter set was applied(λex, 575.5-620.5 nm; λem>630 nm). The Liquid Crystal Tunable Filter (LCTF) was programmed to acquire image cubes from λ=630-700 nm with an increment of 10 nm per image. Fluorescence intensity measurements were calculated as average intensity over the surface area by using Cri Maestro software and ImageJ software.

### Example 1

### Synthesis and Characterization of Nanohydrogels (NHGs)

### 1.1 Nitrile Containing NHGs Before and After Reduction

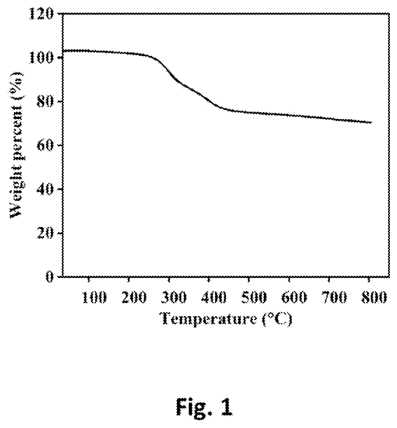

Preparation of NHGs: NIPAM, acrylonitrile (ACN), surfactant (PVP), cross-linker (BIS) and acrylated block copolymer monomers were mixed at different ratios in the presence of an initiator (KPS). It was observed that at constant amounts of NIPAM and ACN, varying the amount of macromonomer resulted in the generation of different NHG sizes, such that lower amounts of macromonomer resulted in largerNHGs (see Table 1). Sizes were well monodispersed, as seen by the polydispersity index. The NHGs of the present invention include nitrile (CN) groups. The presence of nitrile groups in the NHGs allows obtaining NHGs with high number of free amino groups upon reduction. The reduction reactions are performed using borane:THF as reducing agent. Two different procedures were established depending on the size and nature of the NHGs.
1. NHGs of 400 and 200 nm could be precipitated easily using centrifuge. Thus, for these cases the solvent was exchanged from water to THF upon centrifugation and exhaustive washings to remove water (as described above). The smaller NHGs of 50 nm do not precipitate upon centrifuge. These NHGs were reduced according to the method previously described in J. Nanoparticle Res. 2014, 16 (12). https://doi.org/10.1007/s11051-014-2796-1 or in J. Pept. Sci. 2014, 20 (9), 675-679. https://doi.org/10.1002/psc.2664.
2. The method is based on the embedment of the NHGs into a magnetic matrix of magnetite. Briefly, magnetite is generated in the presence of the NHGs that are embedded into the magnetite during the preparation of the magnetite. The obtained matrix embedded NHGs is washed to exchange the solvent taking advantage of the magnetic susceptibility of the matrix. The reaction is then carried out using Borane:THF. Interestingly, even in the presence of magnetite that can be itself reduced; efficient reduction of the 50 nm NHGs is observed (as described above). Direct evidence for the presence of NHGs in the magnetic matrix is provided by thermo-gravimetric analysis (TGA). A pure magnetic matrix has a very small weight loss change upon heating up to 800° C. At the same temperature range, the magnetic matrix embedded NHG's showed a significant weight loss (FIG. 1). The total weight loss for 50 nm embedded NHG's was approximately 30%, which represents the relative quantity of NHG's within the magnetic matrix.
   Table 1 shows size measurements of NHGs before and after reduction. No significant size change was observed when comparing original NHGs with their reduced counterparts. Size analysis at two different temperatures (25° C. and 45° C.) indicate similar thermo-responsivity before and after reduction. The sustained thermo-responsivity indicates that PNIPAM component of the NHGs is mostly unreduced due to steric hindrance at the proximity of the carbonyl groups induced by the i-Propyl nitrogen substitutions. This probably maintains the typical thermos-responsivity observed for PNIPAM.

TABLE 1
Size of NHGs before and after reduction.

| # | | | A | B | C |
|---|---|---|---|---|---|
| Amount of macro monomer (mg) | | | 200 | 400 | 500 |
| Size | Original | 25° C. | 401 ± 4 | 199 ± 10 | 54 ± 4 |
| of NHGs | NHGs | PDI | 0.019 ± 0.001 | 0.058 ± 0.001 | 0.028 ± 0.031 |
| (nm) | | 45° C. | 371 ± 14 | 165 ± 9 | 32 ± 2.5 |
| | | PDI | 0.132 ± 0.229 | 0.265 ± 0.117 | 0.33 ± 0.036 |
| | Reduced | 25° C. | 407 ± 23 | 200 ± 14 | 76 ± 2 |
| | NHGs | PDI | 0.361 ± 0.044 | 0.339 ± 0.024 | 0.869 ± 0.129 |
| | | 45° C. | 361 ± 10 | 172 ± 4 | 41 ± 9.5 |
| | | PDI | 0.355 ± 0.045 | 0.34 ± 0.018 | 0.633 ± 0.288 |

Results are reported as mean±S.D (n=3).
All the polymerizations were performed in 10 ml H₂O containing 170 mg NIPAAM, Acrylonitrile 60 mg (74 μl), 5 mg BIS, 5 mg PVP, and 5 mg K₂S₂O₈ (KPS). (“Block” refers to (Acr)1.1 Jeffamine 1900).

### 1.2. Zeta Potentials

In the zeta potential analysis (Table 2), before reduction it was observed that 400, 200 and 50 nm NHGs disclose a negative zeta potential: −18.6±0.5, −15.4±1.5 and −5.8±0.75 respectively. This is due to the presence of sulfate groups originated from the initiator KPS at the extremities of the polymeric chains. After the reduction process, the zeta potential became positive for all the sizes: 22.6±1, 19.2±2 and 11.6±0.55 respectively. The significant rise of zeta potential demonstrates the presence of new free amino groups on the NHGs. The notable relationship is that heating to 45° C. results in an increased zeta potential for a given NHGs. This effect can be observed for all the size of NHGs. This effect is attributed to the increased density of negative (before reduction) or positive (after reduction) charges caused by NHGs shrinking. It is proposed that the shrunken NHGs have more negative or positive charges at the shear plane, resulting in a higher charge density.

TABLE 2
Zeta potential value of NHGs. Results are reported as mean ± S.D (n = 3)

| # | | | A | B | C |
|---|---|---|---|---|---|
| Size of NHGs (nm) | | | 400 | 200 | 50 |
| Zeta | Original | 25° C. | −18.6 ± 0.5 | −15.4 ± 1.5 | −5.8 ± 0.75 |
| potential | NHGs | 45° C. | −20.5 ± 1 | −17 ± 1 | −8.47 |
| of NHGs | Reduced | 25° C. | 22.6 ± 1 | 19.2 ± 2 | 11.6 ± 0.55 |
| (mV) | NHGs | 45° C. | 25 ± 1 | 23 ± 1 | 14 ± 1.6 |

### 1.3. Fourier-Transform Infrared Spectroscopy (FTIR)

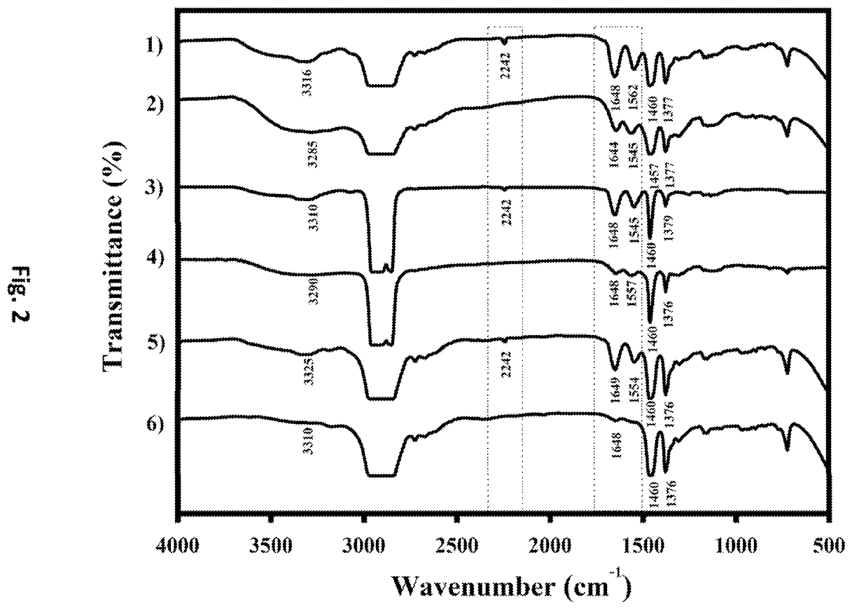

The FTIR spectra of the 400, 200 and 50 nm NHGs are disclosed in FIG. 2. The peak at 2242 cm⁻¹ is due to the C—N stretching vibration of the nitrile group in the starting NHGs. Peaks between 1545 and 1650 cm⁻¹ are attributed to the amide (CONH—R) stretching vibration in the starting NHGs (Navy blue, pink and red plots). Two considerable changes were observed in the FTIR spectra after reduction (Green, blue and black plots): i) the disappearances of C—N stretching vibration due to the formation of reduced NHGs. ii) The amide (CONH—R) stretching vibration (1550-1650 cm⁻¹) intensities are gradually reduced. The FTIR spectra results clearly confirm the formation of new amino groups originated from nitriles and amides reduction as expected.

### 1.4. AFM Analysis of NHGs

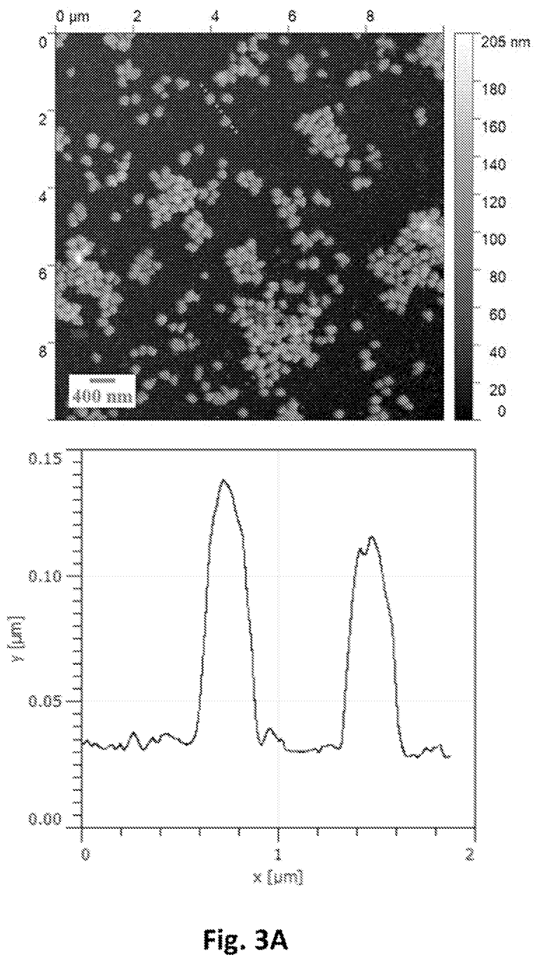

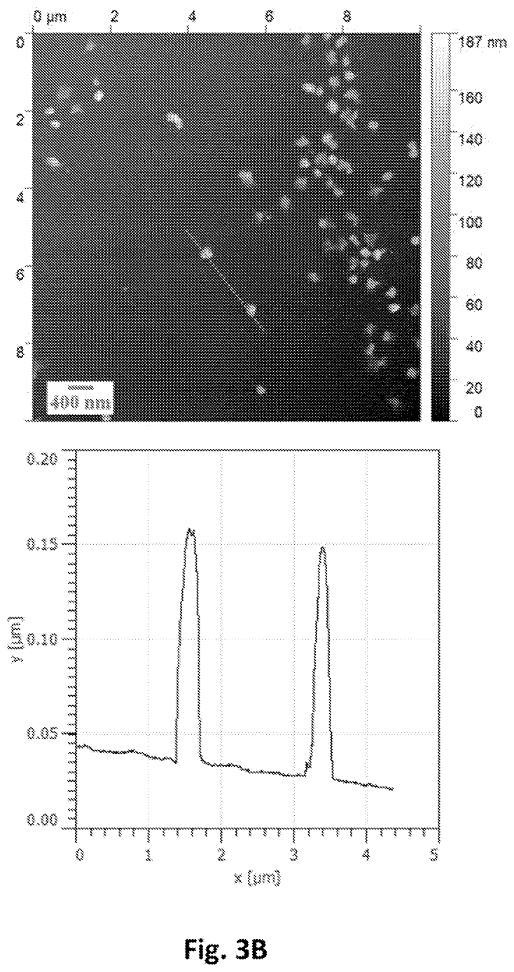

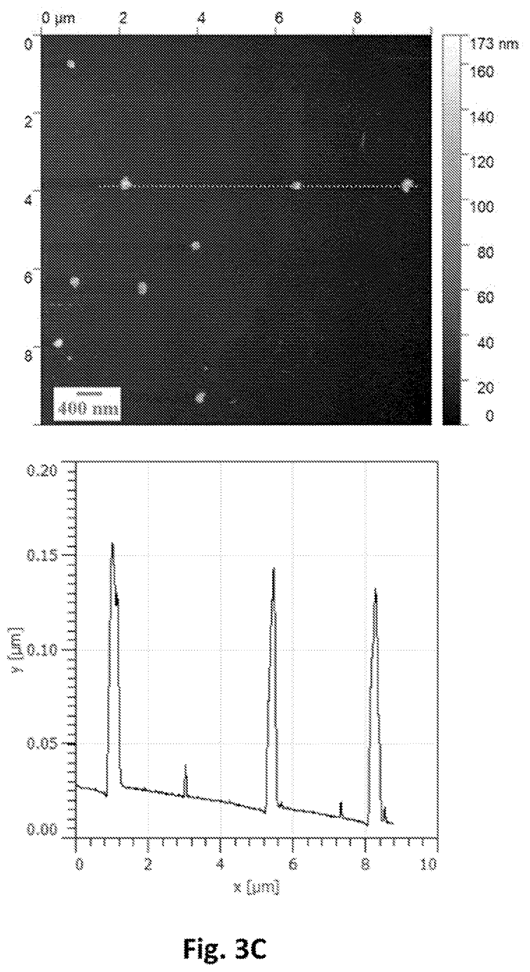

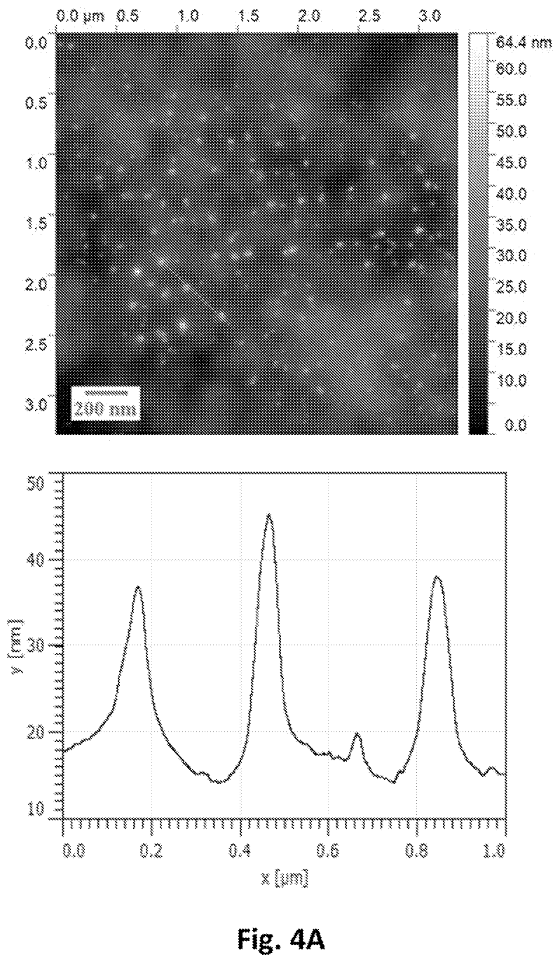

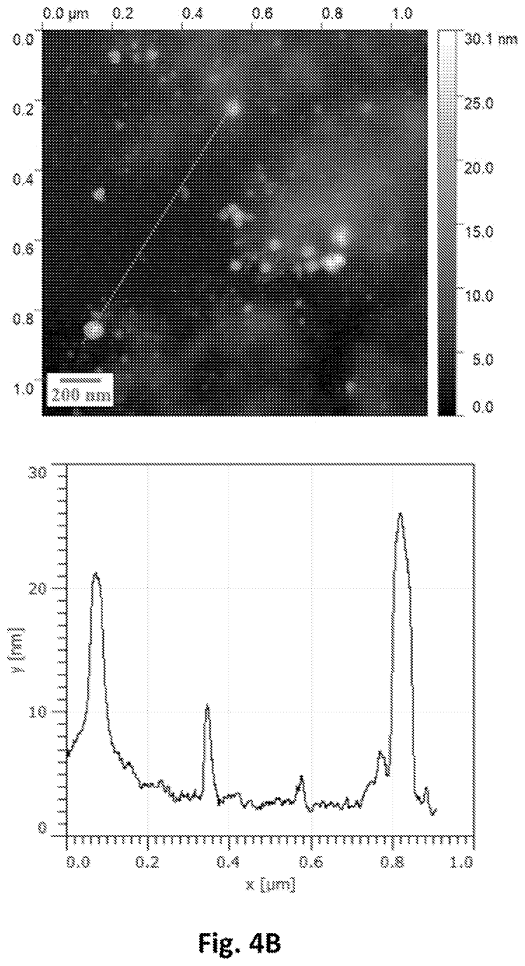

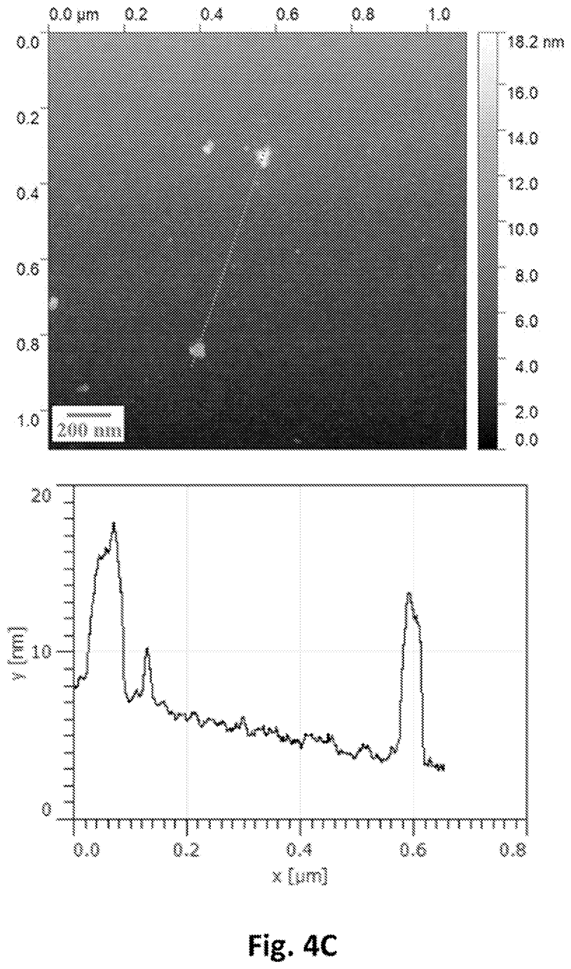

The AFM images of the NHGs show that the effective size of NHGs is smaller than sizes observed using DLS. This is expected due to the dryness of the NHGs when using AFM technic. The NHGs are observed as monodispersed in multilayers when applied at high concentrations, while using at low concentration NHGs are observed as isolated particles on the wafer. The topography of NHGs indicates that the particle size is monodispersed and spherical in shape (FIGS. 3A-C, and FIGS. 4A-C).

### Example 2

### NHGs/DNA Complexes

### 2.1. Gel Electrophoresis

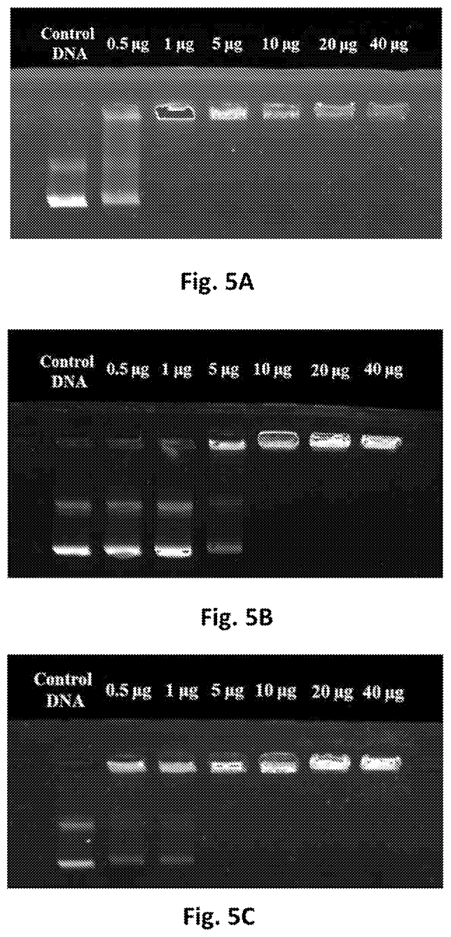

A gel electrophoresis technique (FIGS. 5A-C) was used to determine the DNA loading capacity of NHGs. The experiment was carried out by varying the different concentrations of NHGs to form a complex with a given amount of DNA. The different amounts of NHGs (from 0.5 to 40 μg) were used for the analysis and the DNA (0.5 μg) concentration kept as constant in all the experiments. Naked DNA is used as control. The polyplexes of 400, 200 and 50 nm complexes are formed with a strong interaction starting from 5:1 (NHGs/DNA) weight ratio. The polyplexes of strong interactions were confirmed by UV trans-illuminator.

### 2.2. Size and Zeta Potential of Polyplexes

Polyplexes of 400, 200 and 50 nm NHGs at different NHG/DNA weight ratios were prepared (Table 3). The ratio NHG/DNA were from 1:1 to 40:1. At low NHGs ratio complexes are aggregative (AG* in Table 3), while upon arriving to ratio 5:1 the size of complexes become monodispersed around the size of the naked NHGs. Raising the concentration of NHGs allows the zeta potential values to increase, at maximum ratio of 40:1 in 400, 200 and 50 nm to obtain zeta +22.8, +18.2 and +10.9 mV respectively without size changes. Interestingly, the polydispersity indexes were good for 400 and 200 nm complexes but higher for 50 nm complexes. This might indicate that a single molecule of DNA cannot be enveloped into a single NHG particle, as result of this, aggregated NHGs may be found even at positive zeta potential values

TABLE 3
Size and Zeta potential of polyplexes (400, 200 and 50 nm). AG*- Aggregation

| | NHGs/DNA | | | | | | | | |
|---|---|---|---|---|---|---|---|---|---|
| | (wt/wt) | | | | | | | | |
| # | ratio | 0:1 | 1:0 | 1:10 | 1:1 | 5:1 | 10:1 | 20:1 | 40:1 |
| Polyplexes | Size (nm) | AG* | 405 | AG* | AG* | 410 | 394 | 398 | 403 |
| (400 nm) | 25° C. | | | | | | | | |
| | PDI | 1.000 | 0.186 | 1.000 | 1.000 | 0.341 | 0.242 | 0.302 | 0.316 |
| | Zeta | −10 | 22 | −3.9 | 7.1 | 12.5 | 13.8 | 17.7 | 22.8 |
| | potential | | | | | | | | |
| | (mV) 25° C. | | | | | | | | |
| Polyplexes | Size (nm) | AG* | 230 | AG* | AG* | 255 | 225 | 223 | 227 |
| (200 nm) | 25° C. | | | | | | | | |
| | PDI | 1.000 | 0.242 | 1.000 | 1.000 | 0.372 | 0.259 | 0.267 | 0.292 |
| | Zeta | −8.9 | 19 | −5.7 | 4.9 | 10.7 | 12.4 | 15.7 | 18.2 |
| | potential | | | | | | | | |
| | (mV) 25° C. | | | | | | | | |
| Polyplexes | Size (nm) | AG* | 62 | AG* | AG* | 54 | 65.3 | 67.6 | 56 |
| (50 nm) | 25° C. | | | | | | | | |
| | PDI | 1.000 | 0.772 | 1.000 | 1.000 | 0.942 | 0.842 | 0.741 | 0.686 |
| | Zeta | −9.2 | 12.3 | −8.1 | 3.0 | 7.3 | 8.2 | 10.5 | 10.9 |
| | potential | | | | | | | | |
| | (mV) 25° C. | | | | | | | | |

### Example 3

### In Vitro Experiments

### 3.1. Cell Viability

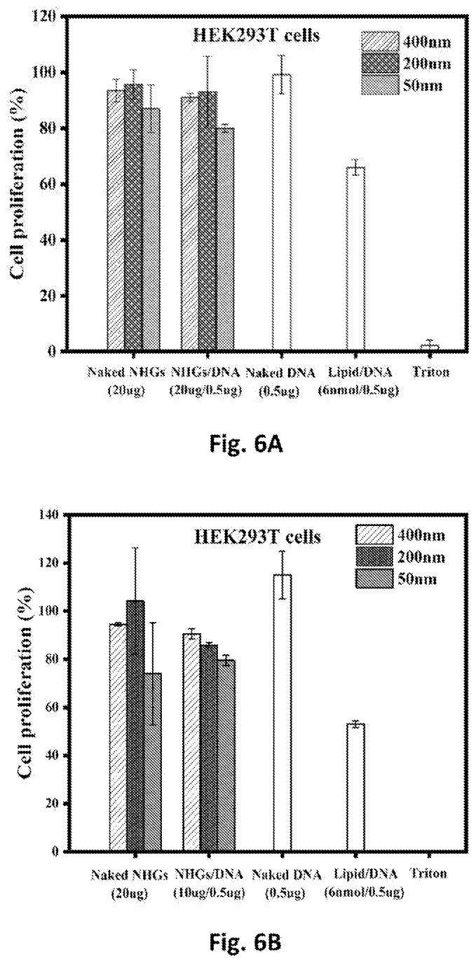

The results (FIGS. 6A-B) demonstrate that polyplexes (400 nm, 200 nm and 50 nm) are devoid of toxicity even at concentrations of 40 μg/ml, in HEK293T cell line for incubation of 2 and 6 days. A standard lipoplex formed with the same DNA using cationic lipid RPR-120535, displayed significant toxicity after 48 h incubation. The cytotoxic effect values were calculated based on quadruplicate at concentration range 10-40 μg/ml of polyplexes. 0.5 μg of DNA kept as constant concentration for all ratios. The cytotoxic effect of NHGs (40 mg/ml), 0.5 μg/ml DNA (GFP), lipoplex (6 nmol/ml), and triton were evaluated as controls.

### 3.2. In-Vitro Transfections

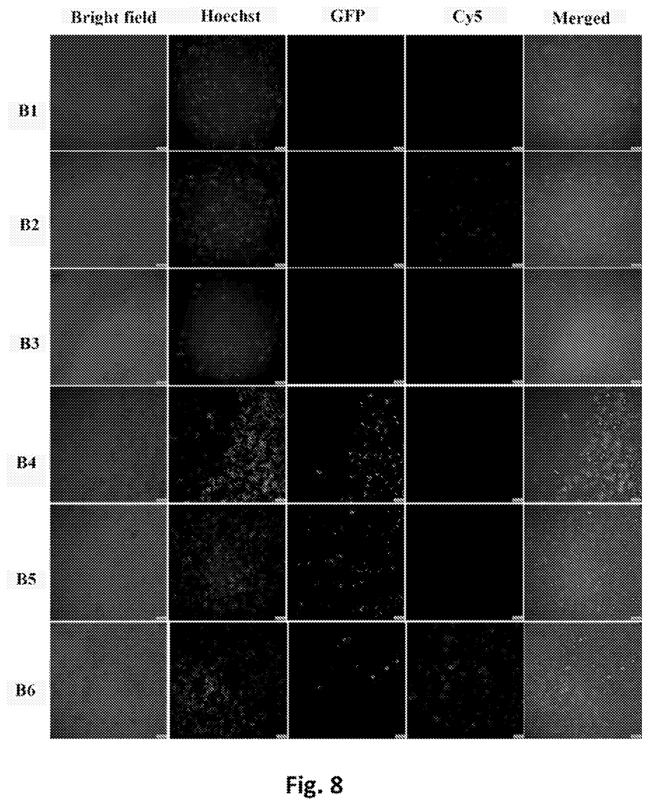

In order to investigate the potential of NHGs to carry DNA, the NHGs were complexed with GFP reporter gene plasmid and incubated with a panel of cell lines. Examples are 5-B6 in FIG. 8). Interestingly, the presence of the complexes inside the cells can be given for HEK-293T and Hela cells. A CY5 labelled NHGs batch has been synthesized in order to follow the cell penetration of complexes.

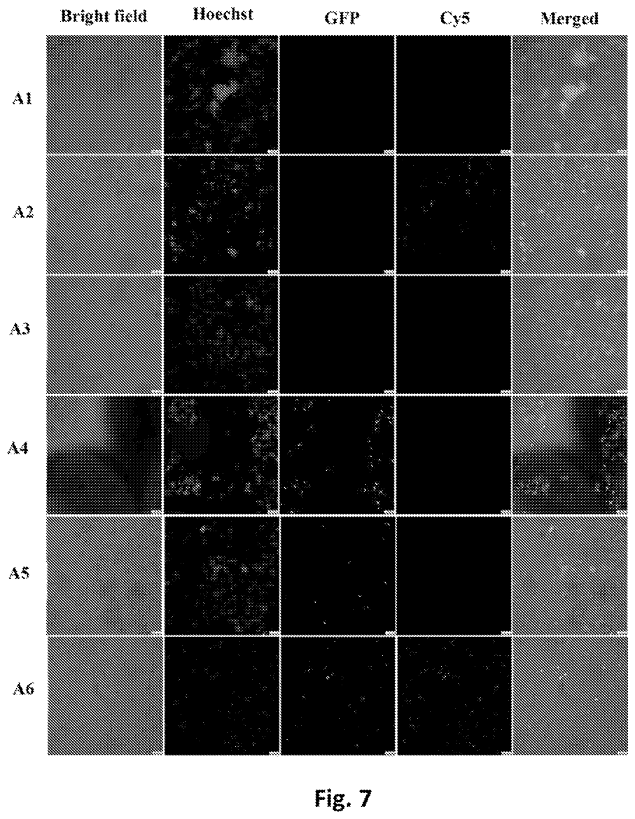

Complexes were formed at different NHGs/DNA ratios. Standard cationic lipid RPR120535 was used at the known optimal transfection lipid/DNA ratio. FIGS. 7 and 8 disclose microscopy pictures for transfection of HEK293 cells after 24 and 48 h incubation respectively, significant transfection for RP120535 (panel A4 in FIG. 7 and B4 in FIG. 8) is observed as compared to NHGs/DNA (panels A5-A6 in FIG. 7 and Bbserved with the fluorescence channel of CY5 both in A6 and B6 in both figures.

The low transfection at 24-48 h is attributed to a slow release of the DNA from the complexes. Thus, longer maturation times for the transfected protein were investigated. Longer maturation times needs refreshing supernatants to keep the cells healthy. Thus, 48 h after incubation, media was exchanged and incubated for extra 4 days. FIG. 9 discloses results obtained after 6 days incubation. Standard RPR120535 lipid/DNA shows a clear decrease in number of cells denoting the significant toxicity of standard transfection agents with a low number of cells that are transfected (see panel C4 in FIG. 9). On the other hand, the cells divided normally both in panels C5 and C6 both displaying a strong fluorescence that indicates high transfection with no cell toxicity. It should also observed that a stock of polyplexes are present inside the cells in panel C6 where CY5 labelled NHGs were used for transfection. This polyplex stock can induce a continuous release of the transgene along many days. Since the turnover of proteins in cells is about 1-2 days, it is suggested that a continuous release and expression of the transgene must be promoted by the presence of the NHGs complexes in the cells. The lack of toxicity of the new NHGs allowed to perform a long-time incubation experiment without interfering with the cell growth. These results open the gates for in vivo sustained expression of transgenes using these NHGs.

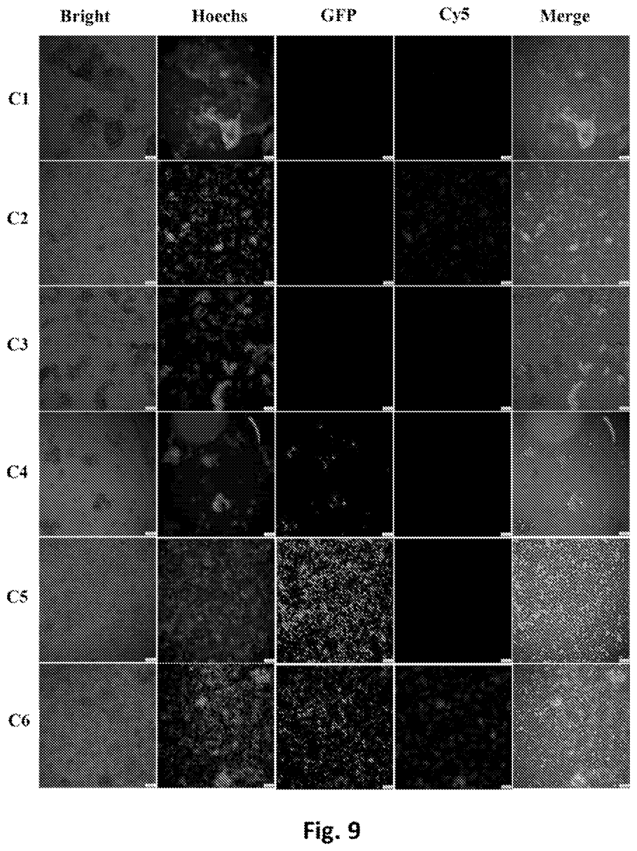

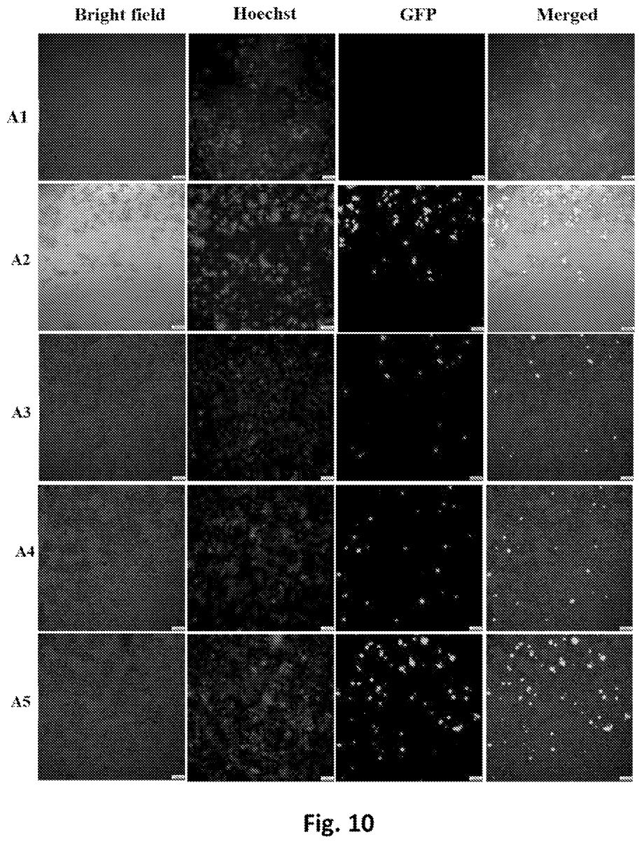

Example is given also for Hela cells with varying ratios of 200 nm NHGs/DNA. Results indicate similar behaviour as for the HK293T cells: after 6 days incubation, lipid complexes were toxic to the cells and NHGs transfected significantly with optimal ratio of 12.5 μg NHGs/0.5 DNA. (FIG. 10).

In order to evaluate and compare transfections using different NHGs sizes, HEK293T and Hela cells were treated also with 400 nm polyplexes. The 400 nm NHGs at various concentrations were complexed with GFP and incubated for 6 days. The findings demonstrate that HEK293T cells are transfected at lower weight ratio (panel A6 in FIG. 11B) and again with observe no toxicity as compared to lipid RPR120535 (panel A3 in FIG. 11A).

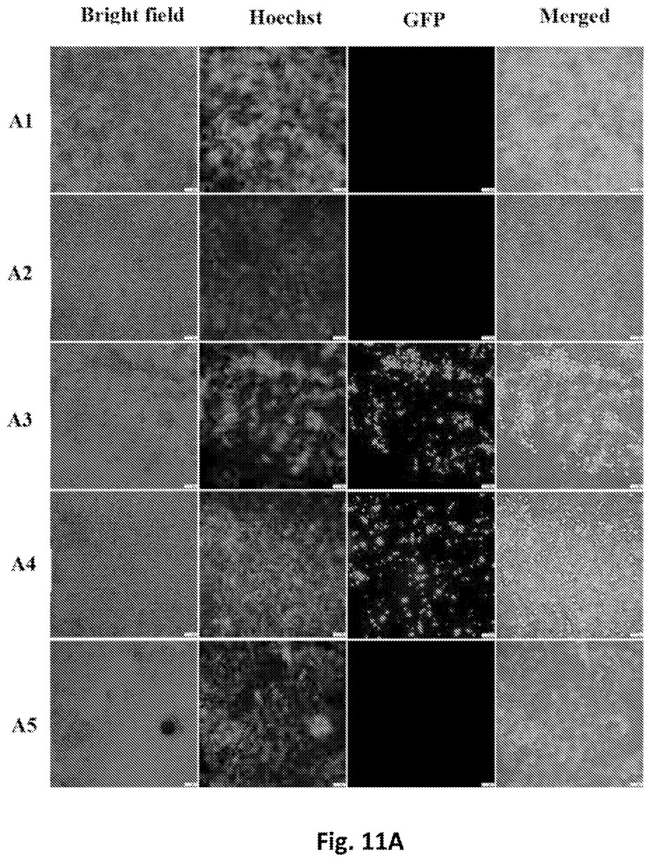

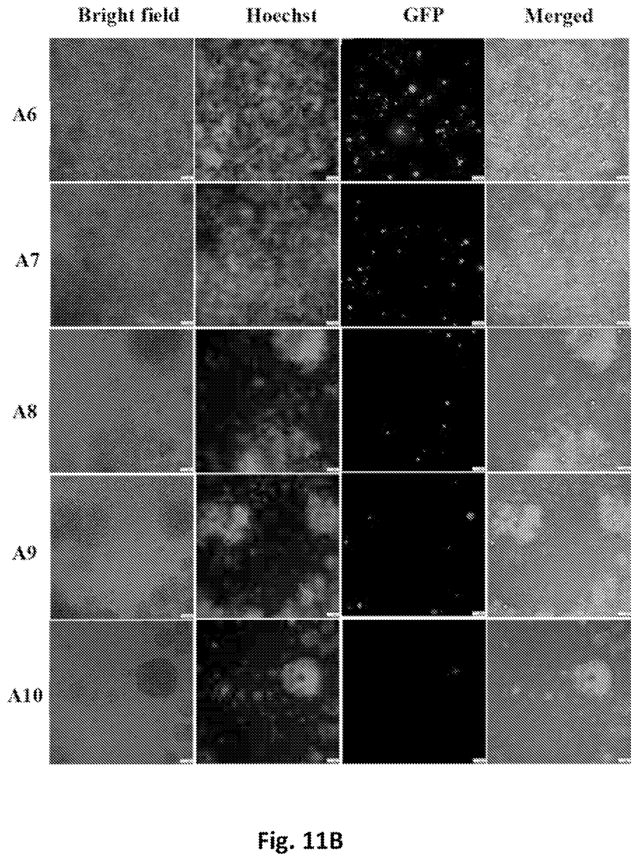

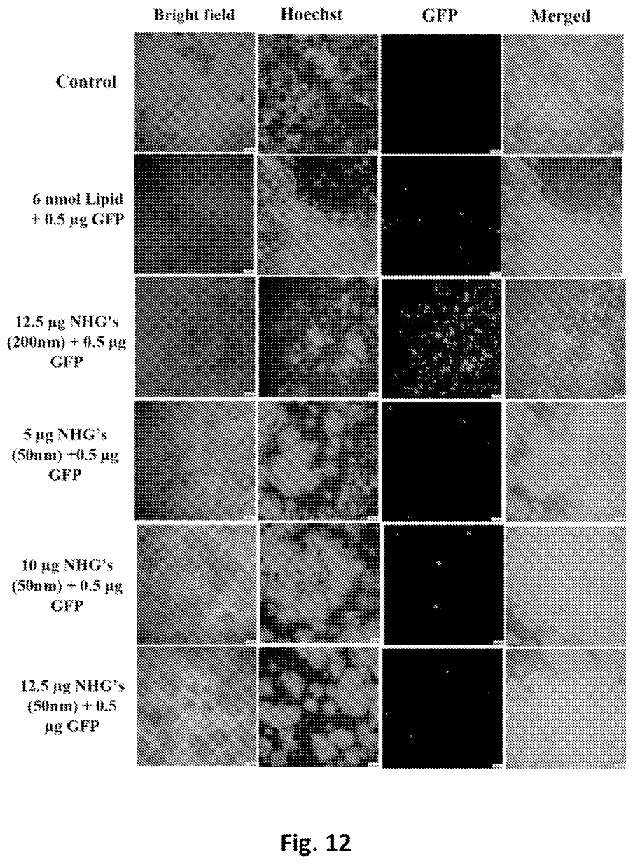

As for the Hela cells, no significant transfection was observed at any weight ratio of the 400 nm NHGs (data not shown). Interestingly, 50 nm polyplexes disclosed a smaller number of transfected cells after 6 days incubation as compared to 200 nm NHGs (FIG. 12).

Overall, it is shown that the 200 nm NHGs display high transfection efficiency at day 6 after incubation with no significant cell toxicity. The 400 nm NHGs display significant trasnfections but at a lower weight ratio with DNA. Finally, 50 nm NHGs resulted in poor transfections at any weight ratio.

### 3.3. Sustained In-Vitro Transfection after Cell-Passing Using 200 nm Polyplexes

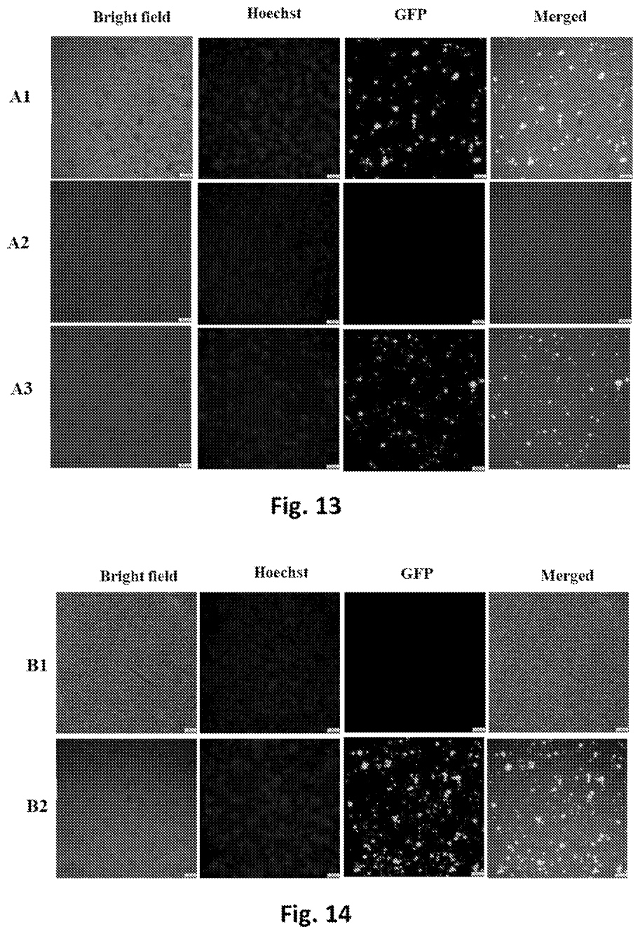

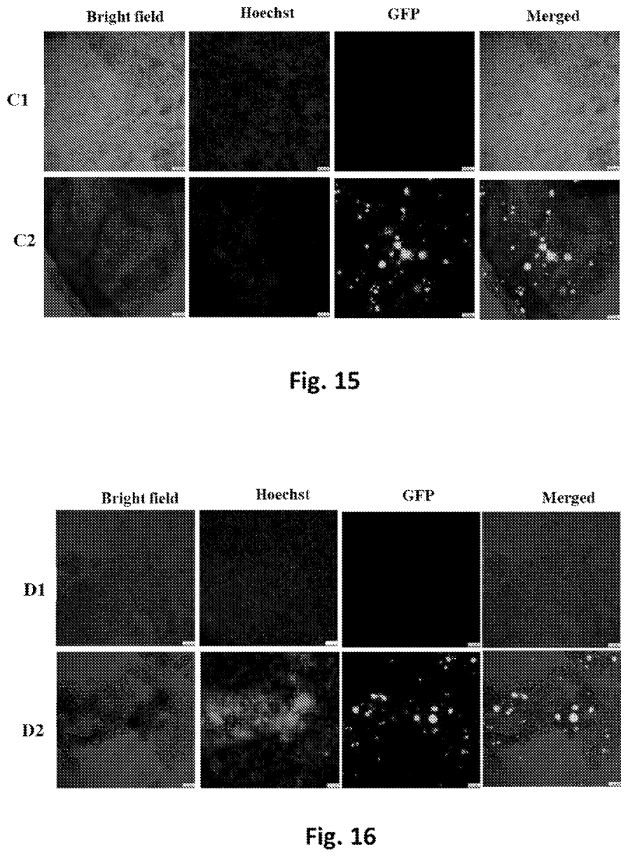

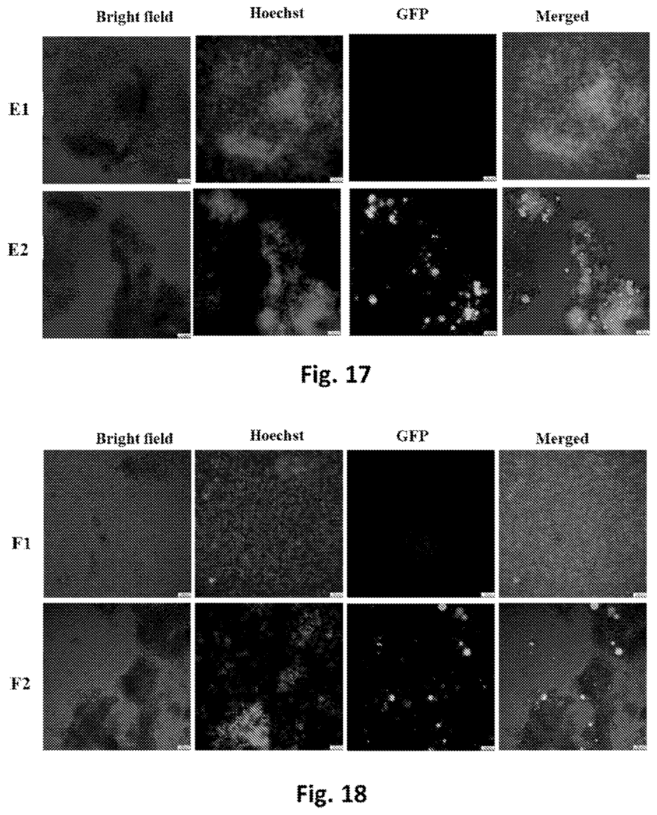

A passing experiment was used to analyse the long-term gene transfection in HEK-293T cell line. The cells were harvested after 6 days incubation with polyplexes (12.5 μg NHGs/0.5 μg DNA) and were divided into 6 equal portion and passed to 6 wells. The wells were analysed one by one at 48 h time intervals starting from day 8 until day 22 after incubation with polyplexes. Untreated cells were passed in the same way and were used as controls. The results reveal that the polyplex can efficiently transfect the cells even two weeks after their passing. Again, this shows evidence that a continuous release of DNA and its expression must be present in the cells since protein turnover is only of about 24 h (FIGS. 13, 14, 15, 16, 17, and 18).

### 3.4. Semiquantitative Analysis of In-Vitro Transfections Using Incucyte Microscope

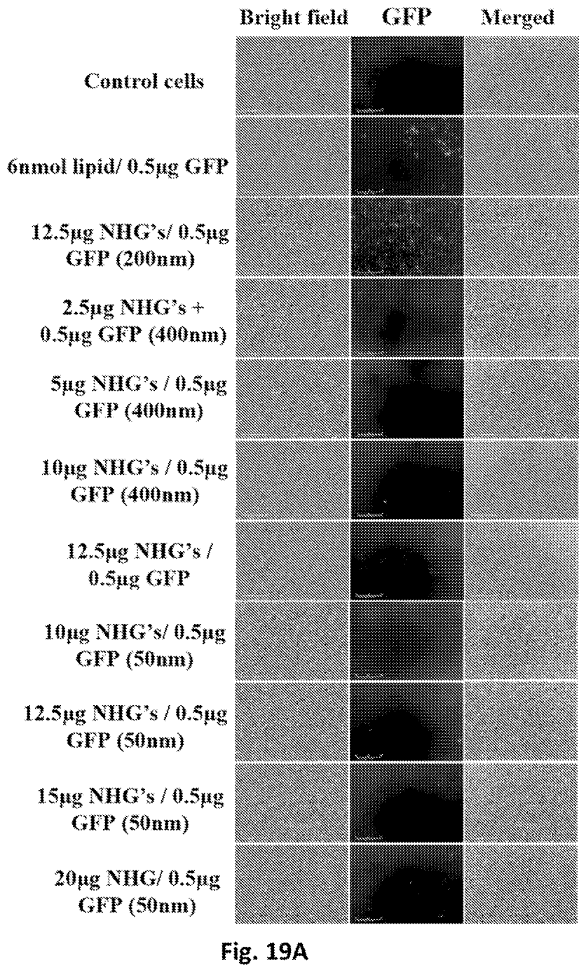

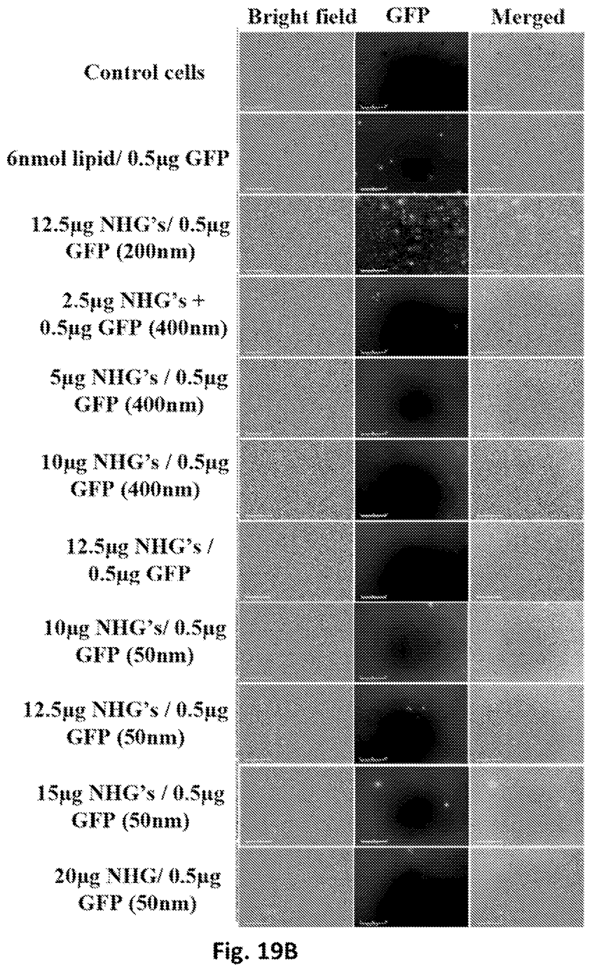

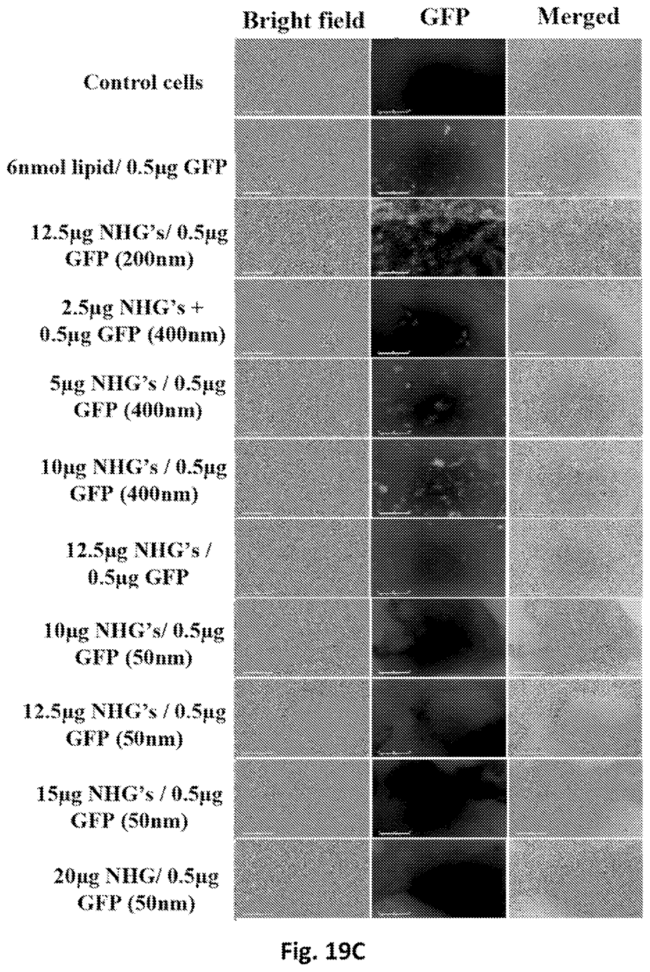

Here only cells are used as negative control and 6 nmol lipid RPR2120535/GFP used as positive control. Various concentrations of NHGs/GFP (400 nm, 200 nm and 50 nm) were used to analyse the gene transfection in the different cell lines: HEK293T, Hela and NIH-3T3. The expression profiles of cell are analysed by the normalized green area/phase area. The results indicate that 200 nm polyplexes have better transfection efficiency as compared to both 400 nm and 50 nm polyplexes (FIGS. 19A-C) in all three cell lines. However, in the 400 nm polyplex few cells get transfected at lower weight ratios. Moreover, no significant transfection was observed for 50 nm polyplex in all three cell lines (FIG. 20).

### Example 4

### In Vivo Transfections

In vitro results demonstrated a long term expression of the marker gene beyond two weeks after incubation without significant cytotoxicity. The in vivo experiment using small mammalian rodent is crucial for evaluating the feasibility of local protein expression as a first step towards the development of a new vaccine methodology or delivery of foreign proteins.

The study aims to study the efficiency of the transfection of a fluorescent gene marker (mcherry plasmid instead of GFP) by local administration of a DNA/NHGs formulation that slowly releases the DNA. Two ways of administration were tested: subcutaneous and intramuscular using two different concentrations of the DNA (20 μg and 40 μg). The expression of the gene was followed by in vivo fluorescence imaging (Maestro) camera during four weeks every 2-3 days after administration.

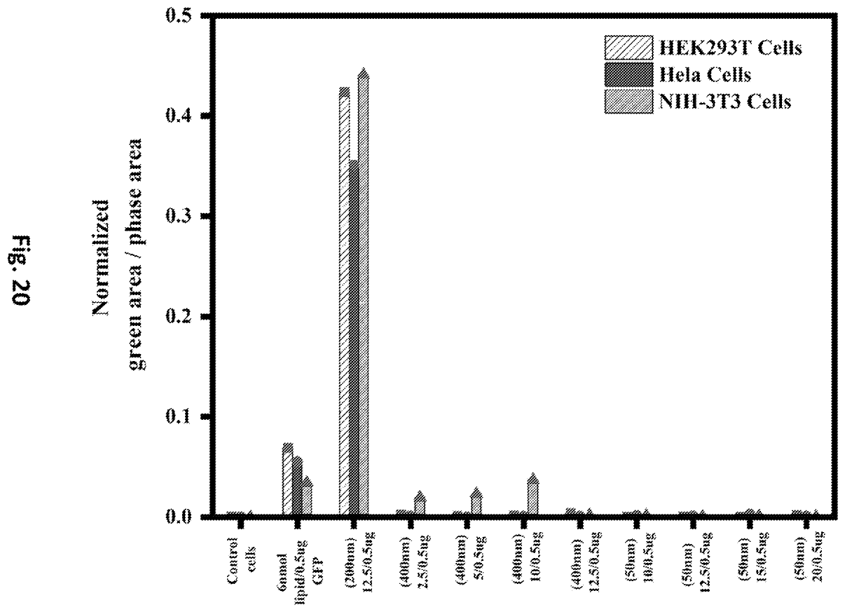

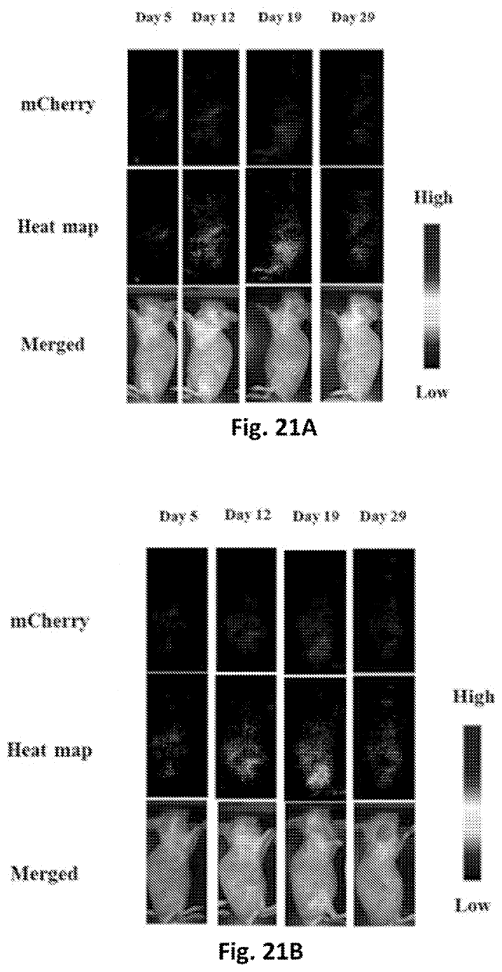

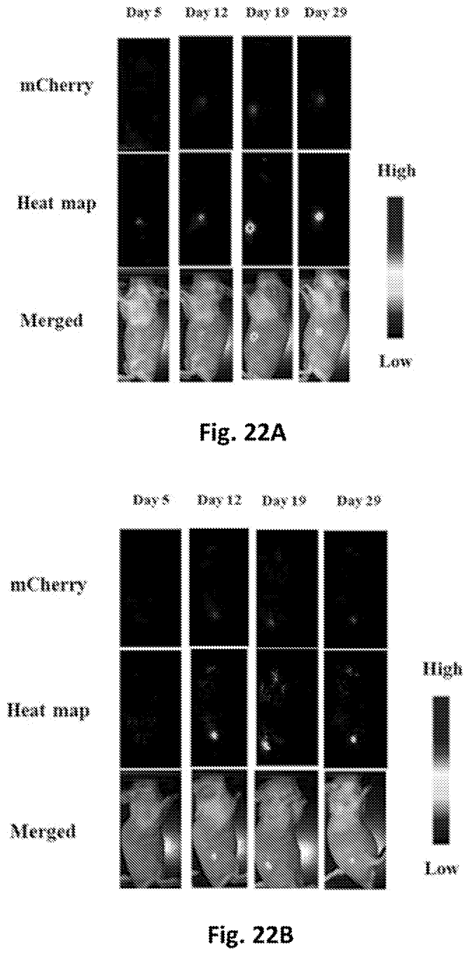

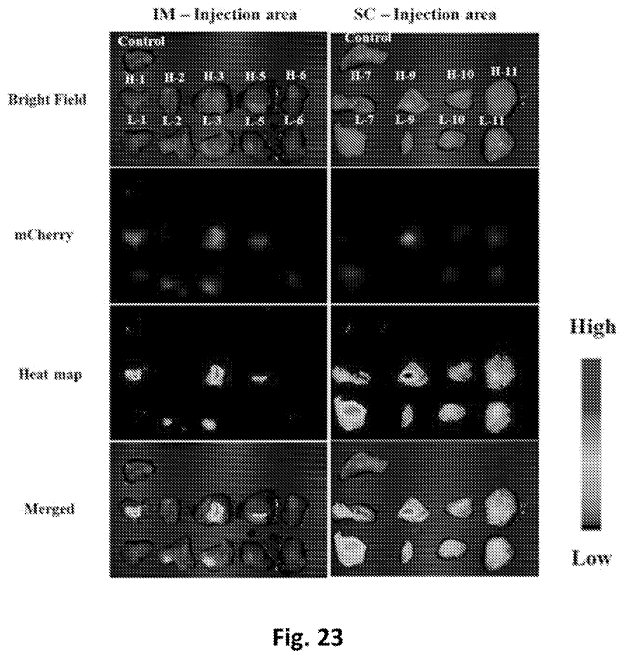

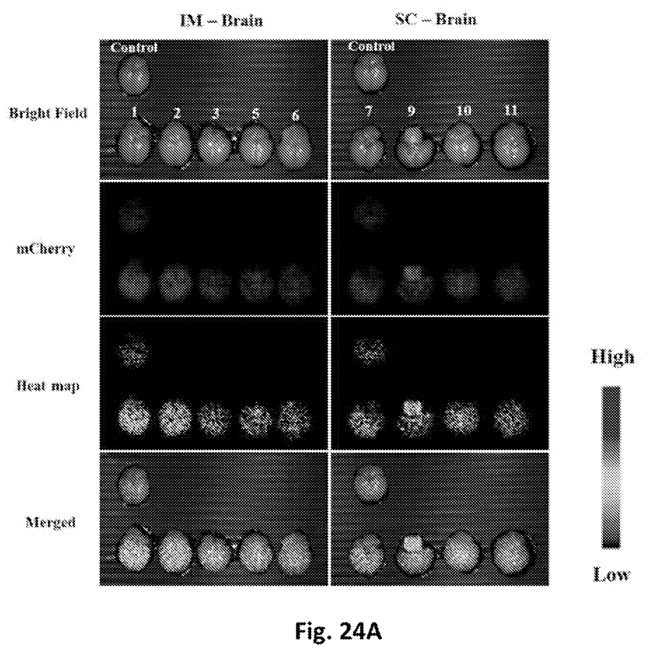

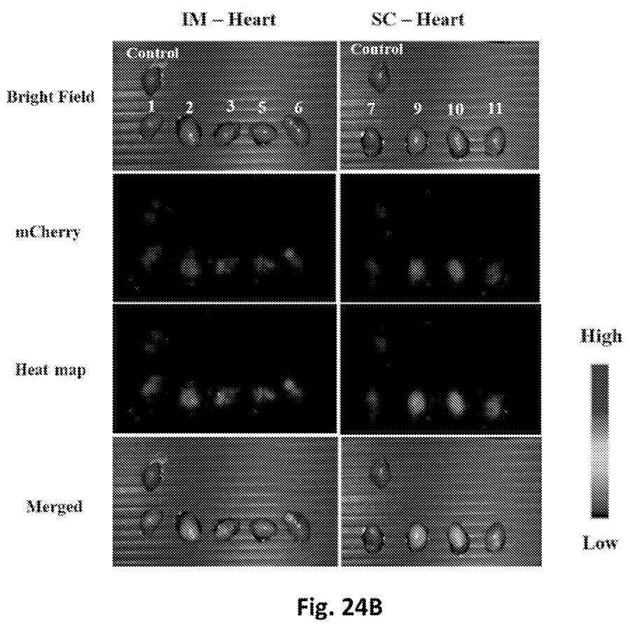

FIGS. 21A-B and FIGS. 22A-B clearly show m-Cherry expression at the site of injection both IM and SC. The expression starts to be observed at day 5 and becomes significant at day 12 until the termination of the experiment. After termination of the experiment, organs were collected and measured under the Maestro camera.

In vivo experiment show that a continuous and long release of DNA plasmid allows sustained expression of a foreign protein at the site of administration. Moreover, DNA can be transported to other organs such as brain and heart and express the foreign protein there.

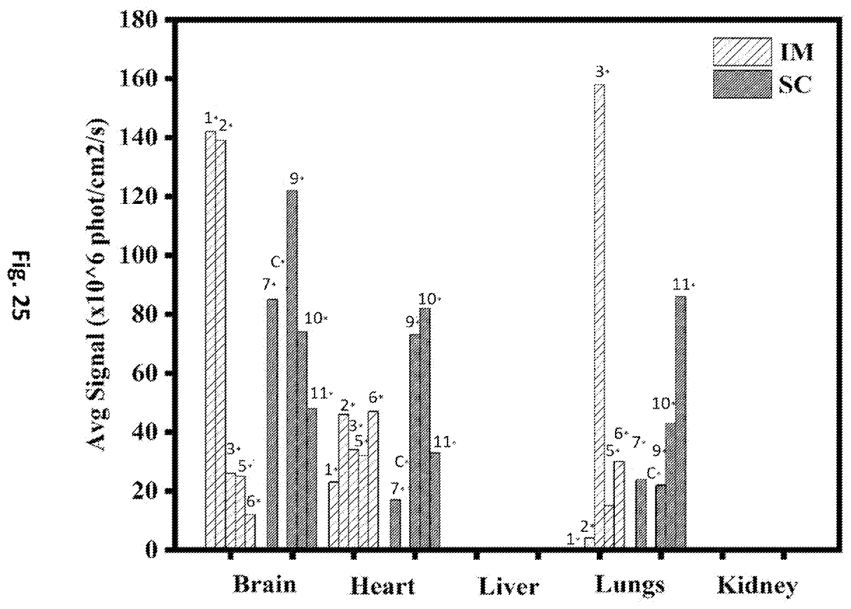

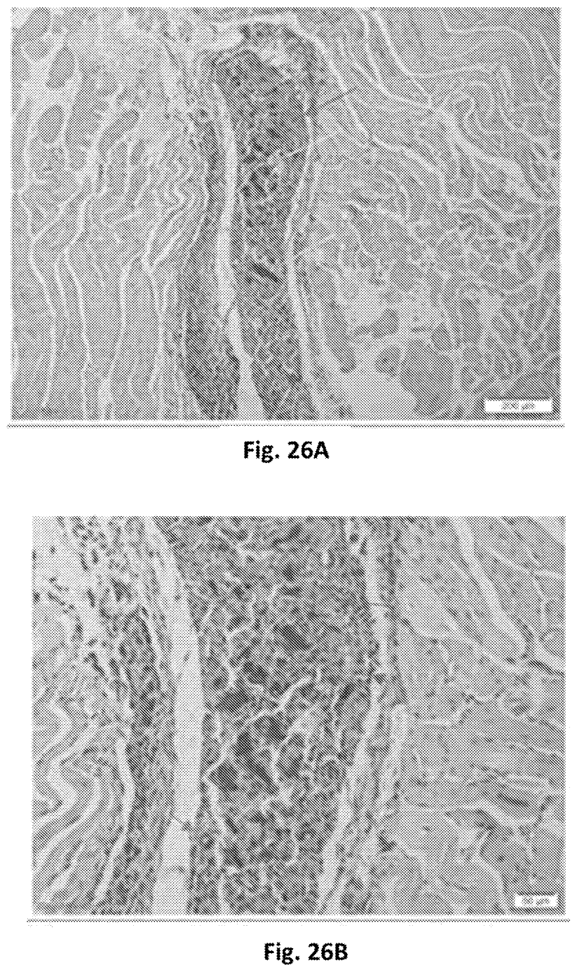

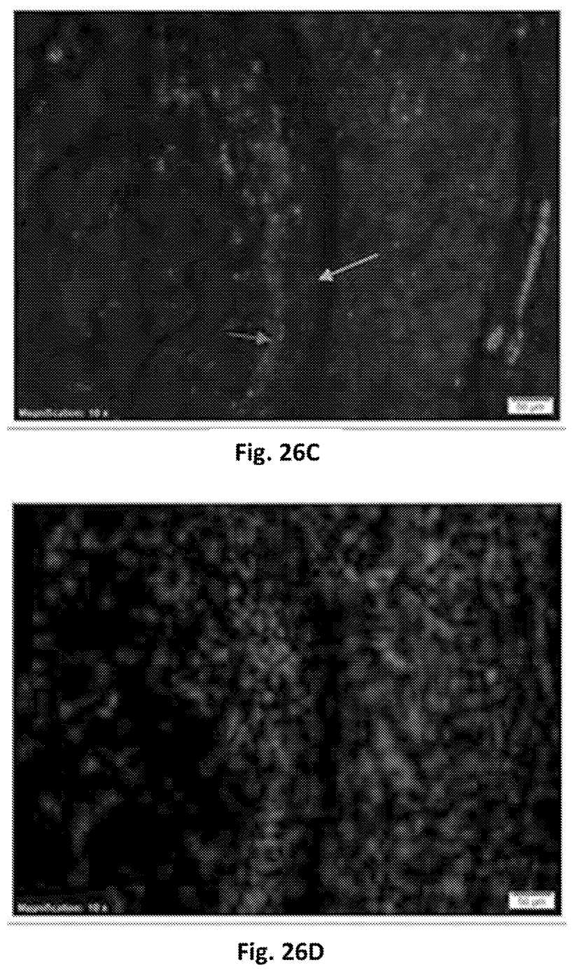

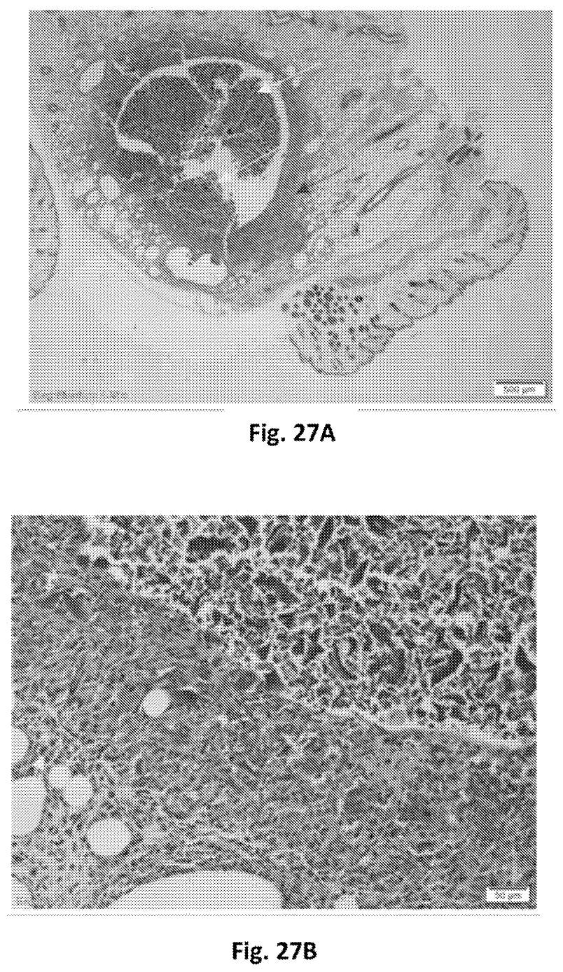

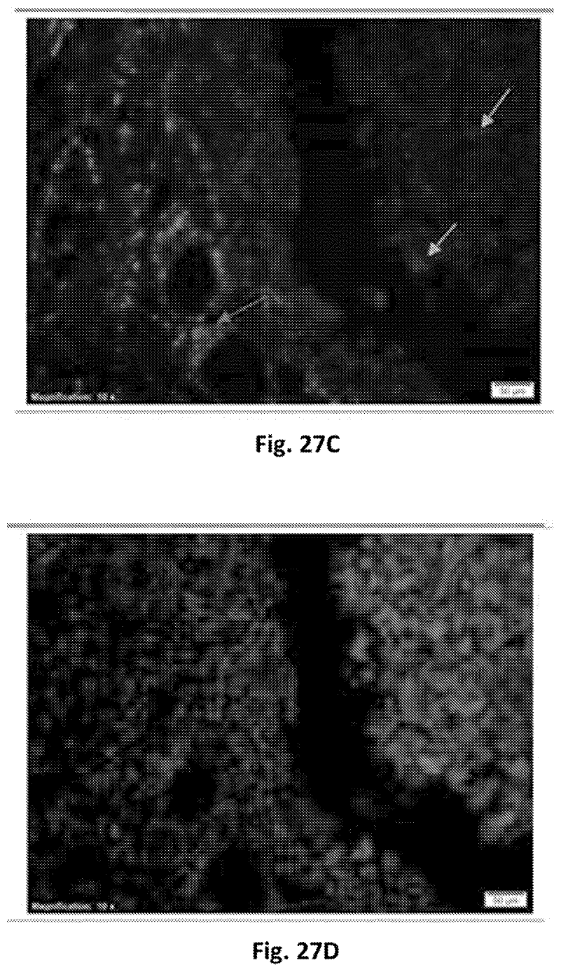

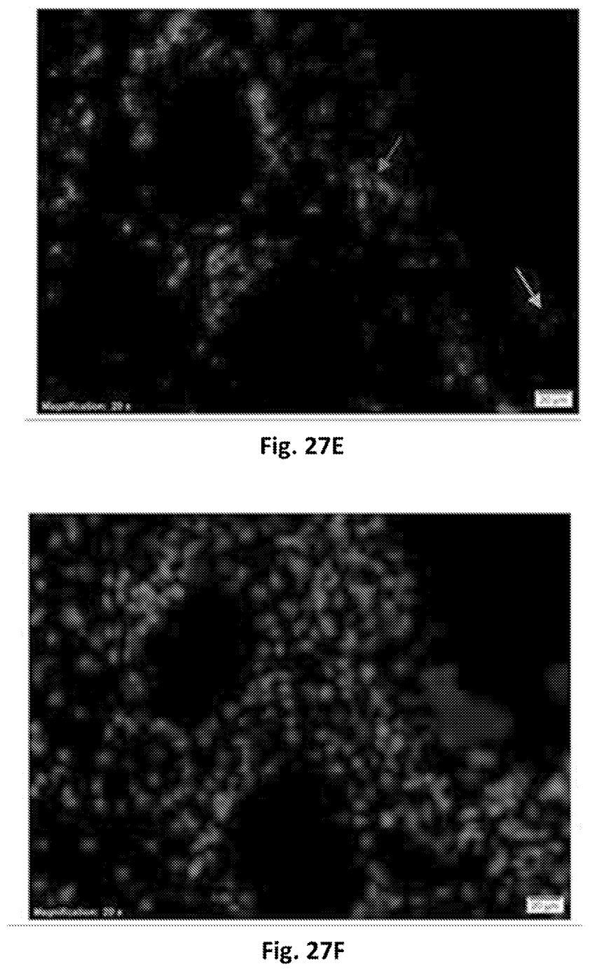

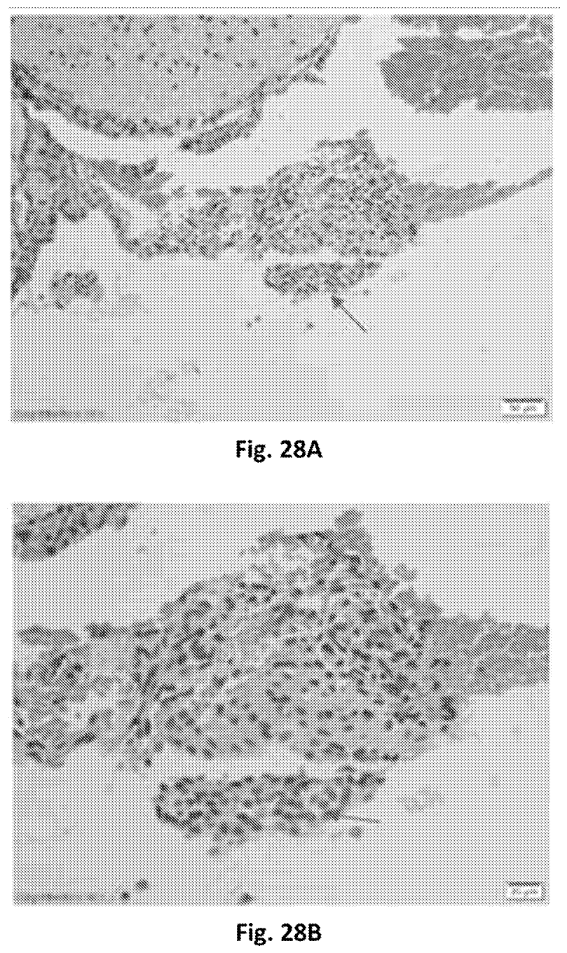

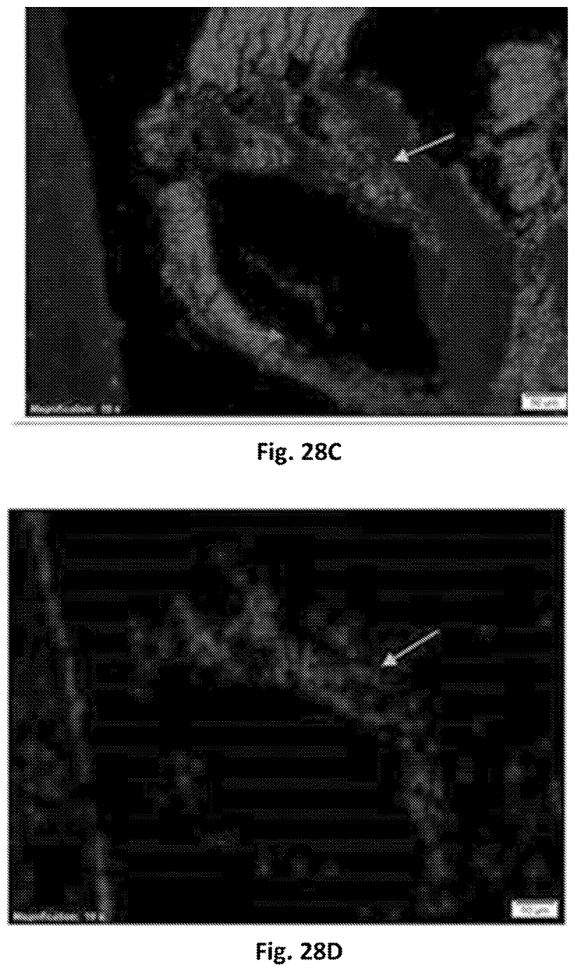

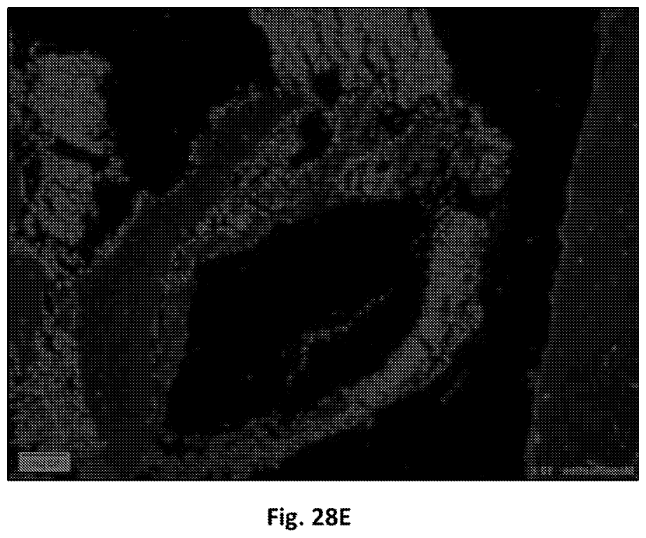

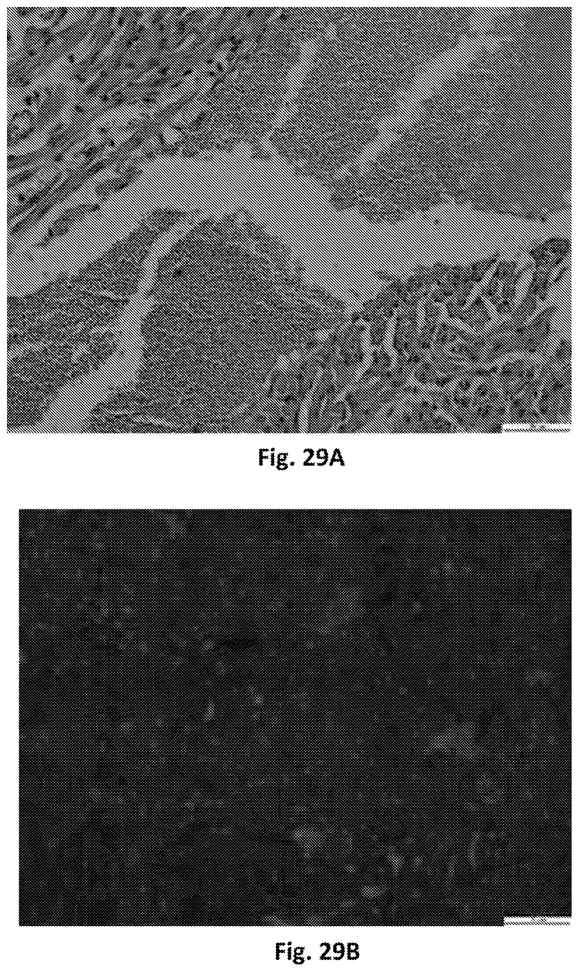

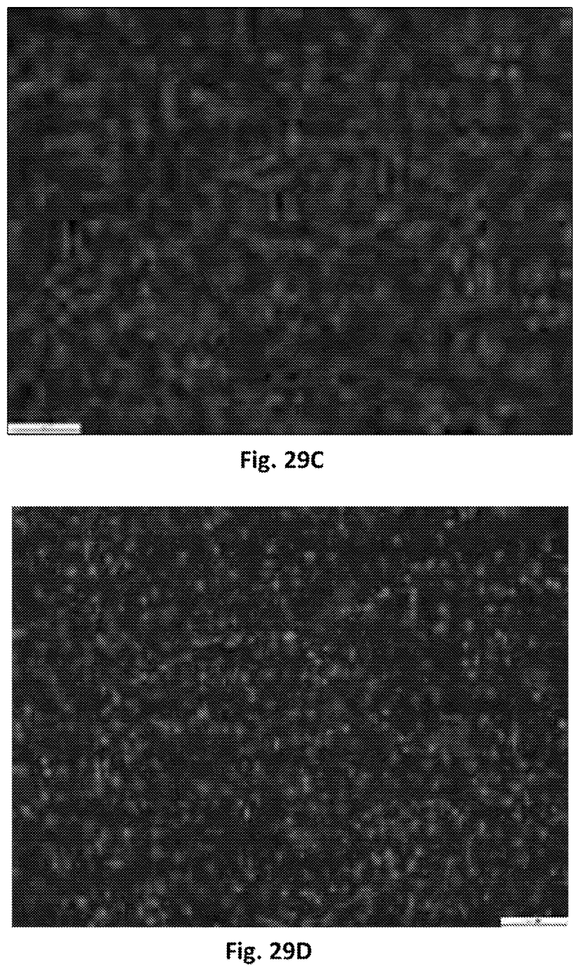

Histological analysis of the administration tissue as well as the brains and heart, show that the transfected cells are mostly those of the immune system. FIGS. 26A-D and 27A-F show the expression of the m-cherry protein at the site of administration both IM and SC, FIGS. 28A-D show the expression of m-cherry in the brain area, and finally, FIGS. 29A-D shows expression of m-cherry in the heart.

### Example 5

### 5.1 Cloning

The gene encoding the mCherry protein was digested using Xbal and Notl, and subsequently cloned into the pGEM-4Z/64A vector.

### 5.2 Synthesis of In Vitro Transcribed mRNA

The mRNA was generated using HiScribe® T7 ARCA mRNA Kit (NEB, Cat #E2065S) according to the manufacturer's instructions, and purified using RNA Clean-Up and Concentration Kit (Norgen, Cat #23600).

### 5.3 Transfections

HEK293T cells were cultured (without antibiotics) in 96-well plate at density of 5000 cells/well and incubated for 24 h to obtain 70-80% of confluence prior to the addition of polyplexes. Before the transfection, the medium was replaced with 100 μl of fresh medium (without serum). The mRNA complexes were prepared as follows: 1 μg mRNA/well mixed in (40 μl) DMEM and appropriate amount of NHG's mixed in (40 μl) DMEM contain no serum. After 5 minutes incubation at room temperature, the diluted mRNA was combined with the diluted NHGs (total volume 80 μl) and incubated for 20 minutes at RT. Complexes were added to each well containing 100 μl of medium without serum and mixed gently by rocking the plate back and forth. After 4 h incubation at 37° C. in CO₂ incubator, 10% FBS was added without removing the complexes. The transgene expression was tested by using IncuCyte (Sartorius) for every 6 h of incubation until day 7. The medium (10% FBS) was replaced after incubated for 48 h.

The cells were harvested after 7 days of incubation, divided into 2 equal portions, and passed to 2 wells. The wells were then analyzed at 6 h time intervals starting from day 7 to 12, and the medium (10% FBS) was replaced after day 9. Untreated cells were passed in the same way and used as controls.

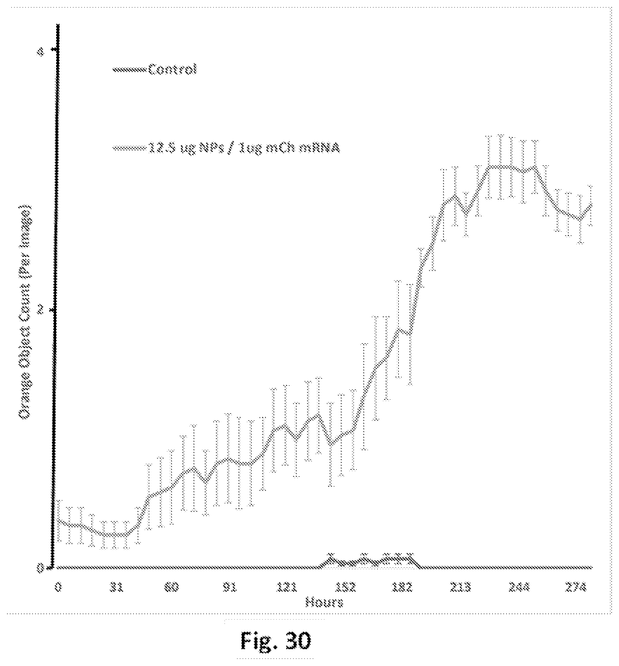

FIG. 30 shows that throughout the 11 days of experiment, the orange object count was significantly higher in HET293T cells transfected with 12.5 μg NHG/1 μg mCherry mRNA compared to control.

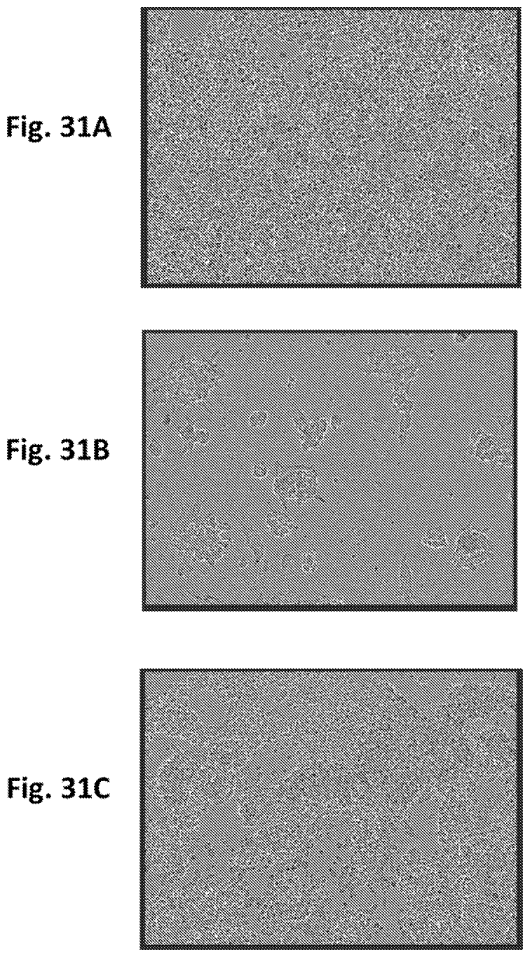

FIGS. 31A-31C show protein expression in HEK293T cells at the 11^{th} day of the experiment. The most prominent protein expression can be seen in HEK293T cells incubated with 12.5 μg mRNA (FIG. 31B) compared to HEK293T cells incubated with 5 μg mRNA (FIG. 31C). No protein expression was evident in the control group, which comprised only HEK293T cells (FIG. 31A FIG. 31A)

Together, these results demonstrate a sustained delivery and expression of mRNA in cells.

1. A process for preparing polymeric monodispersed reduced nanohydrogels (NHGs) comprising:
a) combining monomers, macromonomer, a cross-linker, and a surfactant to obtain a precursor mixture;
b) heating the precursor mixture to a temperature of at least 40° C.;
c) adding an initiator to the precursor mixture to obtain polymeric NHG; and
d) performing an NHG reduction step.

2. The process of claim 1, wherein the cross-linker is bis-acrylamide (BIS).

3. The process of claim 1, wherein the surfactant is Polyvinylpyrrolidone (PVP).

4. The process according to claim 1 wherein the reduction step comprises:
a) preparing a magnetic embedded NHG magnetite or maghemite matrix by combining an NHG with FeCl₃·6 H₂O and FeCl₂·4 H₂O to obtain a reaction mixture, and adding ammonium hydroxide thereto;
b) reducing said magnetic embedded NHG matrix by combining it with an organic solvent-soluble reducing agent to obtain magnetic embedded NHG; and
c) releasing the NHG by dissolving the magnetic matrix in an acid.

5. The process of claim 4, wherein the organic solvent-soluble reducing agent is selected from boric acid, trimethyl borate, and borane-THF complex.

6. The process according to claim 1, wherein the monomers and/or the macromonomer are composed of different chains of jeffamines linked to polymerization groups.

7. The process of claim 6, wherein the polymerization groups are selected from acrylamides, metacrylamides or other substituted acrylamides, or N-isopropyl acrylamide (NIPA), N-diisopropyl acrylamide, N-diisopropyl metacrylamide, acrylonitrile, and any combination thereof.

8. A polymeric monodispersed reduced NHG, whenever prepared by the process of claim 1.

9. The polymeric monodispersed reduced NHG of claim 8, having a monodispersed size of about 20 nm to about 500 nm, preferably from about 50 nm to about 400 nm.

10. A process for preparing a polyplex comprising the NHG of claim 8 and one or more nucleic acids, comprising adding said one or more nucleic acids to the reduced NHG obtained by the process of claim 1.

11. A polyplex comprising a polymeric reduced monodispersed NHG and one or more nucleic acids, whenever prepared according to the process of claim 10.

12. Use of a polyplexes according to claim 11 for transferring genes and/or mRNA into mammalian cells to express endogenous or exogenous protein.

13. A method for the delivery of endogenous or exogenous proteins to the brain or heart comprising using a polyplex according to claim 11 and immune cells as nucleic acid shuttles.

14. A method using polyplexes according to claim 11 to transfer genes and/or mRNA into mammalian cells to express endogenous or exogenous protein, for a use selected from vaccination, treatment of genetic diseases such as hemophilia, cystic fibrosis, spinal muscular atrophy (SMA), Duchesne Myopathy and other genetic diseases, or cancer treatment.
